(19) United States
(12) Patent Application Publication
(10) Pub. No.: US 2004/0248195 A1
(43) Pub. Date: Dec. 9, 2004

(54) Uses of a thyx polypeptide or a nucleic acid encoding such a polypeptide, in particular for screening anti-bacterial or anti-viral compounds

(76) Inventors: Hannu Myllykallio, Les Molieres (FR); Ursula Liebl, Les Molieres (FR); Damien Leduc, Gif Sur Yvette (FR); Gerard Lipowski, Les Ulis (FR); Jean-Louis Martin, Bures (FR)

Correspondence Address: JACOBSON HOLMAN PLLC, 400 SEVENTH STREET N.W., SUITE 600, WASHINGTON, DC, 20004, US

(21) Appl. No.: 10/485,106

(22) PCT Filed: Aug. 2, 2002
(86) PCT No.: PCT/FR02/02795

(30) Foreign Application Priority Data
Aug. 2, 2001 (FR) 0110401
May 3, 2002 (FR) 0205585

(51) Int. Cl. G01N 33/53; C07H 21/04; C07K 14/47

(52) U.S. Cl. 435/7.1; 435/69.1; 435/320.1; 435/325; 530/324; 536/23.5

(57) ABSTRACT

The invention is relative to the various uses of a novel enzyme family, referred to as THYX, capable of catalyzing the synthesis of thymidine 5′-monophosphate in the absence of an active thymidylate synthase enzyme (ThyA).

The invention also relates to reaction media for the thymidylate synthase activity of a THYX polypeptide as well as screening methods implementing said reaction media, as well as kits for implementing such methods.

### FIELD OF THE INVENTION

The present invention relates to the field of enzymes involved in the DNA synthesis, and more specifically to the synthesis of an intermediary compound, thymidine 5′-monophosphate (dTMP), required for producing thymidine 5′-triphosphate (dTTP) constituent for the DNA molecule.

It is also relative to various uses of a new enzyme family, referred to as THYX, capable of catalyzing the synthesis of thymidine 5′-monophosphate in the absence of an active thymidylate synthase (ThyA) enzyme.

The invention also relates to reaction media for the thymidylate synthase activity of a THYX polypeptide as well as screening methods implementing said reaction media, as well as kits for implementing such methods.

### PRIOR ART

Deoxythymidylate, unlike other deoxynucleotides, is not directly produced by a ribonucleotide reductase. Thymidine 5′-monophosphate (dTMP) is disclosed in the state of the art as being the final product of methylation of uridin 5′-monophosphate (dUMP), said methylation being catalyzed by a thymidylate synthase of the THYA type, as well in eukaryotes as in bacteria (Carreras and Santi, 1995). The thymidylate synthase THYA, which is also expressed in mammals, has been contemplated as a potential target of DNA synthesis inhibiting compounds, being useful more particularly for treating colorectal cancer (Papamichael, 2000).

A simplified illustration of the dTMP production within the cell through dUMP methylation is shown in Scheme 1 hereinunder.

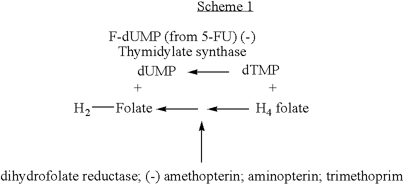

In Scheme 1, there is illustrated the synthesis of dTMP in bacteria and all the eukaryotes through reducing methylation of dUPM under the action of thymidylate synthase THYA.

Another component, essential for dTMP synthesis, is the dihydrofolate reductase enzyme (DHFR), the catalytic activity of which is necessary for recycling dihydrofolate through the formation of an active cofactor, the CH₂H₄ folate, acting as a methyl donor. The thymidylate synthase could be inactivated by a fluoro-dUMP and the DHFR can be inactivated more particularly by amethopterin and aminopterin and trimethoprim, being folate derivates (see Scheme 1).

Many research works done on the synthesis route of the pyrimidine compounds all showed that the intracellular synthesis of dTMP could only be catalyzed by a single enzyme, the thymidylate synthase coded by the thyA gene (see Annual. Reviews on Biochemistry, 1995, vol. 64:721-762; Nature Reviews Mol. Cell. Biol., 2001, vol. 2, 147-151).

Moreover, studies performed on thymidylate synthase inhibitors for treating advanced colorectal cancer point out that <<a significant feature of the nucleotide metabolism is the duplication of the metabolic routes; the inhibition of any enzyme can be bypassed through one or more alternative routes. However, a noticeable exception to this rule is the thyA thymidylate synthase, which is an unavoidable enzyme representing the only way to add a methyl group in position 5 of the pyrimidine cycle in the de novo synthesis of thymidine (Papamichael, 1999).

### SUMMARY OF THE INVENTION

Surprisingly, it has been shown according to the invention that a polypeptide family, referred to as THYX, having a structure completely distinct from the thyA gene coded polypeptides, are able to synthesize dTMP within the cells.

In particular, it has been shown according to the invention that cells with a gene coding a THYX polypeptide have a thymidylate synthase activity, in the absence of thyA gene. It has also been shown that introducing a copy of a thyX gene, coding a THYX polypeptide within an auxotrophic cell for the thymidine could enable to restore, within such a cell, the capacity to de novo synthesize dTMP and finally the thymidylate.

It has also been shown according to the invention that the thyX genes can also be found in the genome of numerous bacteria, bacteriophages and bacterial viruses or eukaryotes, whereas they are absent from the mammalians' genome, more particularly from the human genome.

The characterization according to the invention of a new synthesis route of dTMP by means of the THYX protein family has made available, for the first time, to those skilled in the art the numerous applications directly derived therefrom and which are set forth herein. The various uses of a THYX polypeptide or a nucleic acid coding a THYX polypeptide set forth herein are technically linked by virtue of the common functional and structural features of the THYX polypeptides as hereinunder defined.

An object of the invention is the use of a THYX polypeptide comprising the following amino acid sequence:

X₁HR(X)₇S, wherein:

X₁ represents the amino acid R (Arginine or Arg) or K (Lysine or Lys), and

(X)₇ is a chain with seven consecutive amino acids wherein each X represents, independently from each other, any one of the 20 naturally occurring amino acids,

in an in vitro synthesis method for the thymidine 5′-monophosphate (dTMP),

S represents the Serine amino acid or Ser according to the one letter code in accordance with the international nomenclature.

Preferably, X₁ represents the amino acid R.

Preferably, the THYX polypeptide is selected amongst polypeptides comprising the amino acid sequences SEQ ID N^{o}1 to SEQ ID N^{o}37.

The invention is also relative to using a nucleic acid coding a THYX polypeptide such as defined hereinabove for producing said THYX polypeptide.

Preferably, the nucleic acid is selected amongst nucleic acids comprising the nucleotidic sequences SEQ ID N^{o}44 to SEQ ID N^{o}64.

The invention also relates to using a THYX polypeptide such as defined hereinabove in a method for screening thymidylate synthase inhibiting compounds, more particularly anti-bacterial or anti-viral compounds.

It is also relative to using a nucleic acid coding a THYX polypeptide such as defined hereinabove in a method for screening thymidylate synthase inhibiting compounds, including anti-bacterial or anti-viral compounds.

Another object of the invention is also the use of an antisense oligonucleotide specifically hybridizing with the messenger RNA coding a THYX polypeptide in order to in vitro inhibit the DNA synthesis within a bacterium or a virus.

It is also relative to using a nucleic acid coding a THYX polypeptide such as defined hereinabove as a selection marker for a genetic recombination event.

It also relates to using a specific probe or nucleotidic primer for a nucleic acid coding a THYX polypeptide in order to detect a bacterium or a virus, more particularly a pathogenic bacterium or virus for mammals, and more specifically for man, as well as a set or kit for detecting such a bacterium or such a virus, comprising a specific probe or nucleotidic primer for a nucleic acid coding a THYX polypeptide.

Additionally, the Applicant's works relating to the ThyX activity have made it possible to identify the enzyme mechanism and thereby optimize the reaction conditions enhancing the importance of adding some compounds within the reaction medium.

The aim of the invention is therefore the use of such media and the application thereof.

### DESCRIPTION OF THE FIGURES

FIG. 1 illustrates an alignment of amino acid sequences of various THYX polypeptides.

In the left column is listed the name of the organisms from which each of the amino acid sequences is derived.

The numbers identify the order number of the amino acid at the beginning of the corresponding lineage, within the THYX polypeptide sequence of the organism.

FIG. 2A illustrates immuno-imprint gels made on cellular extracts obtained from E. coli X2913 (ΔthyA) bacteria transfected with the ThyX gene of P. abyssi (lanes number 1 and number 2) or E. coli X2913 (ΔthyA) bacteria transfected with the thyX gene of H. pylon (lanes number 3 and number 4).

The transforming E.coli bacteria were cultivated in the absence of Arabinose (lanes number 1 and 3) or in the presence of 0.2% Arabinose (lanes number 2 and 4). The visible bands on the immuno-imprint gels (3 Western blot>>) respectively correspond to the THYX polypeptide of P. abyssi (lane number 2) and to the THYX polypeptide of H. pylon (lane number 4).

FIG. 2B illustrates photographs of Petri dishes being seeded:

on the left part, by an E. coli Chi2193 (ΔthyA) bacterium transformed with the thyX gene of H. pylori; and

on the right part, with E. coli Chi2193 (ΔthyA) bacteria transformed with the thyx gene of P. abyssi.

The left photograph shows the growth results for the transformed E. coli bacterium cultivated on gelose (agar) in a M9 minimum medium added with 0.2% Arabinose. Only the E. coli bacteria transformed with the thyX gene of H. pylori multiply.

The right photograph shows the same bacteria cultivated in gelose in the presence of a M9 minimum medium in the absence of Arabinose. No bacterial growth is observed, whichever the transfected bacterium is.

FIG. 3 shows a migration on SDS PAGE gel colored with Comassie Blue. The first well corresponds to the PROMEGA Mid-Range marker. The arrows show the molecular weights corresponding to the marker bands. The second well contains the sample of our protein; after being purified on a Ni-NTA column, its band is at about 28 kDa.

FIG. 4 is a Western Blot of the fractions after purification of the protein on a Ni-NTA column, which shows THYX purification.

M=PROMEGA Mid-Range marker,

TF=the first fraction after incubation of the protein with the resin,

L=fraction after washing, and

E1=eluted protein.

FIG. 5 shows biochemical analyses of the ThyX of H. Pylori.

FIG. 6 illustrates the formation activity for dTMP of the ThyX protein of H. pylori.

### DETAILED DESCRIPTION OF THE INVENTION

It has been shown for the first time according to the invention that a polypeptide family, referred to as THYX, distinct from the polypeptide family coded by the thyA genes, has a catalytic activity of the thymidylate synthase type.

The Applicant cloned the thyX gene of H. Pylori in a functional expression vector in Escherichia coli. Such an expression vector has been used for transforming an auxotrophic Escherichia coli strain for thymidine, more specifically the E. coli X2913 (ΔthyA572) strain, the genetic material of which is precisely characterized and wherein the thyA gene is deleted. The results being set forth in the examples show that the expression of the thyx gene which has been artificially introduced into the X2913 (ΔthyA) strain has made it possible to restore the ability of E coli to de novo synthesize dTMP. Because of the large knowledge gathered on the features of the E. coli genome as well as on the normal synthesis route of dTMP via the expression of the THYA thymidylate synthase, the so-obtained results show that the expression product for the thyx gene of H. Pylori directly overcomes the production deficiency of the THYA polypeptide in such a bacterial organism.

Through a sequence homology study, it is shown according to the invention that the polypeptides having sequences homologous to the THYX polypeptides of Helicobacter pylori (SEQ ID n^{o}21) were also coded by the genome of numerous bacteria, Archaebacteries bacteriophages as well as in some viruses.

Surprisingly, it is also shown according to the invention that all the bacteria, Archae bacterie and viruses having in their genome a copy of a thyX gene do not have simultaneously the thyA gene, previously known as the only gene able to perform the dTMP synthesis. Such organisms having the thyx gene in the absence of the thyA gene do not include either any copy of the tdk gene coding the thymidine kinase required for an intracellular incorporation of exogenous thymidine which is subsequently converted within the cell into dTMP, with the noticeable exception of the Mycobacterium tuberculosis bacterium. The simultaneous presence of both the thyA and thyX genes in M. tuberculosis is probably due to a gene transfer event.

The mutual exclusion of the thyA and thyx genes in the genome of the above-mentioned organisms clearly shows that thyX compensates for the thymidylate synthase function that is no longer ensured in the absence of thyA. Additionally, only the thyA gene is systematically absent from the genome of bacteria having the thyxgene, while other genes involved in the nucleotide metabolism are undiscriminately present or absent from the genome of such organisms.

It is also shown according to the invention that thyA deficient bacteria wherein the thyX gene is present are mostly pathogenic bacteria for mammals. Are more specially to be mentioned Campylobacterjejuni causing poisonings food intoxications, Helicobacter pylori being a causal agent for ulcers, Rickettsia prowazekii being a causal agent for typhus, Borrelia burgdorferi being involved in Lyme's disease, Treponema pallidum being the causal agent for syphilis, bacteria of the Chlamydiae genus which are compulsory intracellular pathogens as well as eukaryotic DNA viruses such as Chorella virus.

It has also been shown according to the invention that bacteria wherein the thyX gene has been inactivated become auxotrophic for thymidine, i.e. they only multiply if exogenous thymidine is added to the culture medium.

All the proteins belonging to the THYX family having a newly identified thymidylate synthase activity according to the invention share in common the structural and functional features as indicated hereinunder.

Structural Features

a) The thyx genes code, all without exception, a THYX polypeptide comprising the following amino acid sequence:

X₁H R(X)₇ S, wherein:

X₁ represents the amino acid R (Arginine or Arg) or K (Lysine or Lys), and

(X)₇ is a chain with seven consecutive amino acids wherein each X represents, independently from each other, any of the 20 naturally occurring amino acids.

Preferably, X₁ represents the amino acid R.

X₁ represents K, particularly for the THYX polypeptide coded by the Roseophage S101 genome.

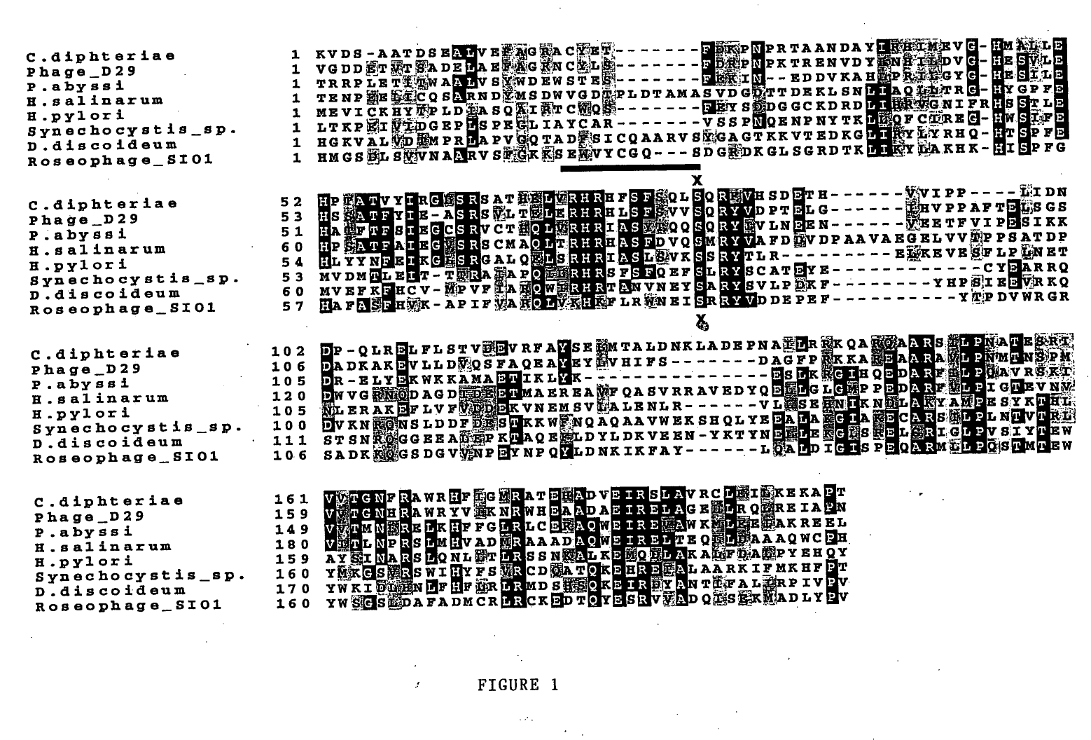

b) As clearly shown by the alignment of the amino acid sequences of the THYX proteins from various origins illustrated in FIG. 1, the THYX polypeptides have a preserved serine amino acid residue. Moreover, the results from the mutagenesis experiments made on the thyX gene of Helicobacter pylori, as set forth in the examples, show that the preserved serine amino acid is indispensable for the catalytic activity of the THYX polypeptide, since substituting respectively a cysteine or alanine residue for the serine residue leads to the production of a polypeptide being unable to compensate for the THYA polypeptide deficiency in the E. coli X strain of 2913 (ΔthyA).

c) The THYX polypeptides do not have any cysteine amino acid residues being preserved in their sequences, unlike the polypeptides coded by the thyA genes where the preserved cysteine residue has a nucleophilic essential part in the methylation reaction catalyzed by the THYA polypeptides.

d) Using the BLAST software (version 2.0) with the default parameters and then the Psi-BLAST module in order to perform iterative cycles of homology research with the THYX polypeptide sequence of H. Pylori as <<lure>>, no target sequence of thyA thymidylate synthase has been selected, showing the lack of homology between thya and thyX genes.

Functional Features

a) A THYX polypeptide according to the invention is able to catalyze the oxidation of methylene tetrahydrofolate into tetrahydrofolate, which is a feature of the catalytic activity of the thymidylate synthase type, and shows that the THYX polypeptides belong to the enzyme class of the thymidylate synthase type;

b) the growth of microoganisms transformed by a thyX gene could be inhibited by a high trimethoprim concentration, being an inhibitor specific for the dihydrofolate reductase; the fact that only high trimethoprim concentrations inhibit THYX suggests that there are functional differences between the metabolic formation routes of thymidylate where are respectively involved thyA and thyX;

c) bacteria wherein the thyX gene has been inactivated become auxotrophic for thymidine;

d) a THYX polypeptide according to the invention is only catalytically active in the presence of a co-factor of flavin type. It is to be noted that the polypeptides coded by the thyA genes, previously known as being the only genes coding enzymes of the thymidylate synthase type, are active without requiring the presence of flavin.

Are included in the polypeptides belonging to the THYX polypeptide family having structural and functional features as defined hereinabove the THYX polypeptides comprising the amino acid sequences SEQ ID N^{o}1 to SEQ ID N^{o}37.

The THYX polypeptides of the sequences SEQ ID n^{o}1 to SEQ ID n^{o}37 were made available to the public specially through their publication in data bases of amino acid sequences.

An object of the invention is the use of a THYX polypeptide comprising the following amino acid sequence:

X₁ HR(X)₇ S, wherein:

X₁ represents the amino acid R (Arginine or Arg) or K (Lysine or Lys), and

(X)₇ is a chain with seven consecutive amino acids wherein each X represents, independently from each other, any of the 20 naturally occurring amino acids,

in an in vitro synthesis method for the thymidine 5′-monophosphate (dTMP).

Preferably, the amino acid X₁, represents the amino acid R.

In order to achieve the in vitro synthesis of dTMP by means of a THYX polypeptide according to the invention, those skilled in the art could more particularly refer to the examples herein, wherein the thymidylate synthase activity of the THYX polypeptide of Helicobacter pylori (SEQ ID N^{o}21) is shown in cellular extracts through the detection of the oxidation reaction of the methylene tetrahydrofolate compound.

Preferably, the above-mentioned use is characterized in that the THYX polypeptide is selected amongst polypeptides comprising the amino acid sequences SEQ ID N^{o}1 to SEQ ID N^{o}37.

According to another aspect, the above-mentioned use is characterized in that the THYX polypeptide is selected amongst polypeptides comprising the amino acid sequences SEQ ID N^{o}1 to SEQ ID N^{o}37.

The THYX polypeptide with amino acid sequences SEQ ID N^{o}5 is coded by a gene derived from the Dictyostelium discodideum organism which was disclosed in 1989 by Dynes and Firtel, and referred to as <<Thy1>> by these authors.

However, DYNES and FIRTEL explicitly excluded that the Thy1 gene of Dictyostelium discoideum, could code a thymidylate synthase. The biosynthesis route for thymidine by Dictyostelium discoideum, which is still not known heretofore, was obviously unknown in 1989, as well as were also unknown the molecular bases causal for the thymidine autotrophy. In addition, the Dictyostelium discoideum organism has not yet been the subject of systematic sequencing studies of its genome. A fortiori, in 1989, no data was available regarding the characterization of the genetic material of such organism, which is still an insurmountable technical barrier for identifying the direct functional part of a mutation, more particularly a mutation leading to an alteration of a metabolic route as complex as that of nucleotides, in particular the thymidine nucleotide. In fact, DYNES and FIRTEL did not characterize the nature of the mutation in Dictyostelium discoideum. The function of the DNA insert of the clone allowing for complementing the organism so as to restore the autotrophy through thymidine was totally unknown. Additionally, since 1989, the numerous research teams for studying the cellular biosynthesis route of dTMP continued to gather experimental results showing that the thymidilate synthase coded by the thyA genes would be the only synthesis route for dTMP (above-mentioned D. PAPAMICHAEL The Oncologist, 1989).

The invention is also relative to using a nucleic acid coding a THYX polypeptide such as defined hereinabove with a view to producing said THYX polypeptide for implementing it in the various uses of a THYX polypeptide as disclosed herein.

Starting from amino acid sequences SEQ ID N^{o}1 to SEQ ID N^{o}37 and/or nucleotidic sequences SEQ ID N^{o}44 to SEQ ID N^{o}64, those skilled in the art are able to detect, isolate, clone and characterize any nucleic acid coding a THYX polypeptide such as defined hereinabove, for example synthetizing nucleotidic probes specific for a nucleic acid coding the X₁ HR(X)₇ S or RHR(X)₇ S peptide.

For making such probes, those skilled is the art could adapt their sequence depending on the use of the codon for a given organism. The detection of a thyX gene could be achieved through hybridation on a DNA gel (<<Southern Blot>>) as well as through PCR amplification, for example using the above defined probe as a nucleotidic primer.

For example, the thyX gene of H. Pylori could be isolated by means of the nucleotidic primers with sequences SEQ ID N^{o}38 and SEQ ID N^{o}39 and the thyx gene of P. abyssi could be isolated by means of the nucleotidic primers with sequences SEQ ID N^{o}40 and SEQ ID N^{o}41. The thyX gene of Campylobacter jejuni could be isolated by means of the nucleotidic primers with sequences SEQ ID N^{o}42 and SEQ ID N^{o}43.

Preferably, the nucleic acid coding a THYX polypeptide is selected amongst the nucleic acids coding a THYX polypeptide comprising one of the amino acid sequences SEQ ID N^{o}1 to SEQ ID N^{o}37.

According to another aspect, the nucleic acid is selected amongst the nucleic acids coding a THYX polypeptide consisting in one of the amino acid sequences SEQ ID N^{o}1 to SEQ ID N^{o}37.

Advantageously, the nucleic acid is selected amongst the nucleic acids comprising the nucleotidic sequences SEQ ID N^{o}44 to SEQ ID N^{o}64.

Amongst the THYX polypeptides able to be implemented according to the invention are included the THYX polypeptides having at least 95% amino acid identity with a THYX polypeptide selected amongst sequences SEQ ID N^{o}1 to SEQ ID N^{o}37.

Amongst the nucleic acids coding a THYX polypeptide able to be implemented according to the invention is included a nucleic acid having at least 95% nucleotide identity with a nucleic acid selected amongst nucleotidic sequences SEQ ID N^{o}44 to SEQ ID N^{o}64.

Identity between two Nucleic Acids or between two Polypeptides

For the purpose of the present specification, the expression <<nucleotidic sequence>> is used for undiscriminately referring to a polynucleotide or a nucleic acid. The expression <<nucleotidic sequence>> encompasses the genetic material itself and hence, is not restricted to the information regarding the sequence thereof.

According to the invention, a first nucleic acid having at least 95% identity with a second reference nucleic acid, would have at least 95%, preferably at least 96%, 97%, 98%, 98.5%, 99%, 99.1%, 99.2%, 99.3%, 99.4%, 99.5%, 99.6%, 99.7%, 99.8% or 99.9% nucleotide identity with said second reference polynucleotide, the identity percentage between two sequences being determined as described hereinunder.

According to the invention, a first polypeptide having at least 95% identity with a second reference polypeptide, would have at least 95%, preferably at least 96%, 97%, 98%, 98.5%, 99%, 99.1%, 99.2%, 99.3%, 99.4%, 99.5%, 99.6%, 99.7%, 99.8% or 99.9% amino acid identity with said second reference polypeptide, the identity percentage between two sequences being determined as described hereinunder.

The “identity percentage” between two nucleotide or amino acid sequences, as meant in the present invention, could be determined comparing two optimally aligned sequences, through a comparison window.

The part of the nucleotidic or polypeptidic sequence in the comparison window could hence comprise additions or deletions (for example “gaps”) as compared to the reference sequence (which does not comprise such additions or such deletions) so as to obtain an optimal alignment of the two sequences.

The percentage is calculated by determining the number of positions where an identical nucleic base or a amino acid residue is observed for the two sequences (nucleic or peptidic) to be compared, then dividing the number of positions where there is an identity between the two amino acid bases or residues to be compared, by the total number of positions in the comparison window, then multiplying the result by one hundred so as to obtain the sequence identity percentage.

The optimal alignment of sequences for the comparison could be achieved with a computer using known algorithms.

Preferably, the sequence identity percentage is determined using the BLAST software (version BLAST 2.06 dated September 1998), exclusively using the default parameters.

A nucleic acid having at least 95% nucleotide identity with a nucleic acid according to the invention encompasses “variants” of a nucleic acid according to the invention.

As used herein a nucleic acid “variant” according to the invention means a nucleic acid differing from the reference nucleic acid through one or more substitutions, additions or deletions of a nucleotide, compared to the reference nucleic acid. A variant of a nucleic acid according to the invention could be from natural origin, such as a naturally occurring allelic variant. Such a variant nucleic acid could also be a non-naturally occurring nucleic acid obtained, for example, using mutagenesis techniques.

Generally, the differences between the reference nucleic acid and the “variant” nucleic acid are reduced such that the reference nucleic acid and the variant nucleic acid have very similar nucleotidic sequences and in numerous regions identical. The nucleotidic modifications present in a variant nucleic acid could be silent, meaning that they do not affect the amino acid sequence which could be coded by such a variant nucleic acid.

The nucleotide modifications in such a variant nucleic acid could also result in substitutions, additions or deletions of one or more amino acids in the sequence of the polypeptide that could be coded by such a variant nucleic acid.

More preferably, a variant nucleic acid according to the invention comprising an open reading phase, codes a polypeptide maintaining the same biological function or the same biological activity as the polypeptide coded by the reference nucleic acid.

Most preferably, a variant nucleic acid according to the invention and comprising an open reading phase, codes a THYX polypeptide maintaining the catalytic activity of a thymidylate synthase, which could more particularly be detected by the ability of the THYX polypeptide to oxidize the methylene-tetradihydrofolate in vitro as well as to restore the ability of a Thya thymidylate synthase deficient bacterium or eukaryote cell to synthesize DNA, as described in the examples.

Much more preferably, a variant THYX polypeptide will not comprise any amino acid modification on the X₁HR(X)₇S pattern, and the preserved Serine amino acid is present.

In the THYX polypeptides able to be implemented according to the invention are included the THYX polypeptides coded by a nucleic acid such as defined hereinabove.

Are also included in the definition of a THYX polypeptide according to the invention the THYX polypeptides comprising one or more amino acid substitutions in one of the sequences SEQ ID N^{o}1 to SEQ ID N^{o}37 by an <<equivalent>> amino acid. Are included in the definition of “equivalent” amino acids, the amino acids belonging to the same class, such as the acidic (D and E), basic (K, R and H), non polar (A, V, L, l, P, M, F and W) as well as non charged polar (G, S, T, C, Y, N and Q) amino acids.

Are also within the scope of the invention the polypeptides referred to as “homologous” to any of the THYX polypeptides of the amino acid sequences SEQ ID N^{o}1 to SEQ ID N^{o}37, or the variants thereof.

Such homologous polypeptides have amino acid sequences with one or more substitutions of an amino acid by an equivalent amino acid, as compared to the reference polypeptides.

It is meant by equivalent amino acid according to the present invention, for example the substitution of a residue in the D form for a residue in the L form as well as the substitution of a pyro-glutamic acid for a glutamic acid (E) according to techniques well known to those skilled in the art. By way of illustration, the synthesis of a peptide containing at least one residue in the D form is disclosed by KOCH (1977).

According to another aspect, are also considered as being equivalent amino acids two amino acids belonging to the same class, i.e. two acidic, basic, non polar as well as non charged polar amino acids.

Preferably, the polypeptides according to the invention comprising one or more additions, deletions, substitutions of at least one amino acid maintain their ability to be recognized by antibodies raised against the unmodified polypeptides. Such polypeptides also maintain their thymidylate synthase catalytic activity.

Preferably, a THYX polypeptide or a nucleic acid coding a THYX polypeptide able to be implemented according to the invention is in isolated or purified form.

The term “isolated” as used herein means a biological material which has been removed from its original environment (the environment where it is naturally occurring). For example, a polypeptide or a polynucleotide naturally occurring in an animal or a plant is not isolated. The same polypeptide separated from its natural environment or the same polynucleotide separated from the adjacent nucleic acids wherein it is naturally inserted in the genome of the animal or of the plant is isolated.

Such a polynucleotide could be included in a vector and/or such a polynucleotide could be included in a composition and could yet remain in the isolated state, because the vector or the composition is not its natural environment.

The term “purified” does not require that the material should be present in absolute purity form, excluding the presence of other compounds. It is rather a relative definition. A polypeptide or a polynucleotide is in a purified state after purification of the starting material of at least one order of magnitude, preferably 2 or 3 and more preferably 4 or 5 orders of magnitudes.

Are also within the scope of the invention, THYX polypeptides with sequences SEQ ID N^{o}1 to SEQ ID N^{o} 37 made proteolysis resistant through the introduction of one or more non peptidic links, such as a reduced link (CH₂NH), a retro-inverso link (NHCO), a methylene-oxy link (CH₂—O), a thiomethylene link (CH₂—S), a carba link (CH₂—CH₂), a ketomethylene link (CO—CH₂), a hydroxyethylene link (CHOH—CH₂) as well as a CH═CH link.

In all cases, a THYX polypeptide able to be implemented according to the invention maintains the catalytic activity of a thymidylate synthase, which could be more particularly detected by the ability of the THYX polypeptide to oxidize the methylene-tetradihydrofolate in vitro as well as to restore the ability of a THYA thymidylate synthase deficient bacterium or eukaryotic cell to synthesize the DNA, as disclosed in the examples.

Production of a THYX Polypeptide able to be Implemented According to the Invention

The invention is also relative to a method for producing one of the THYX polypeptides as defined hereinabove, in particular a polypeptide selected amongst the THYX polypeptides with amino acid sequences SEQ ID N^{o}1 to SEQ ID N^{o}37 or of a variant thereof, said method comprising the steps of:

a) inserting a nucleic acid coding said polypeptide into an appropriate vector;

b) cultivating, in an appropriate culture medium, a host cell previously transformed or transfected with the recombinant vector from step a);

c) recovering the conditioned culture medium or lysing the host cell, for example through sonication or osmotic shock;

d) separating and purifying said polypeptide from said culture medium or also from cell lysates obtained in step c);

e) if need be, characterizing the produced recombinant polypeptide.

The THYX polypeptides according to the invention could be characterized through an attachment on an immunoaffinity chromatography column where the antibodies raised against such a polypeptide or against a fragment or a variant thereof have been previously immobilized.

According to another aspect, a recombinant THYX polypeptide according to the invention could be purified through passing on an appropriate plurality of chromatography columns, using methods known to those skilled in the art.

A THYX polypeptide according to the invention could also be prepared using the traditional chemical synthesis techniques, either in a homogenous solution or in a solid phase.

By way of illustration, a THYX polypeptide according to the invention could be prepared using the technique or in a homogenous solution as disclosed by HOUBEN WEYL (1974) or also the solid phase synthesis technique as disclosed by MERRIFIELD (1965a; 1965b).

Methods for Screening Thymidylate Synthase Inhibiting Compounds

As already previously set forth, the thyX genes are found in numerous pathogenic bacteria for mammals, in particular being pathogenic for man, and in some viruses. Additionally, unlike the thyA genes, the thyx genes are not to be found in the mammals' genome. More particularly, the human genome is thyx gene free.

As a result, THYX polypeptides and nucleic acids coding THYX polypeptides are preferred targets for compounds specifically inhibiting the expression of thyx genes or specifically inhibiting the thymidylate synthase activity of THYX polypeptides. Such inhibiting compounds are able to inhibit the DNA synthesis in numerous bacteria, bacteriophages and viruses, more particularly those being pathogenic for mammals, including for man, as well as the multiplication of such bacteria, bacteriophages and viruses, while not causing unwanted effects in such mammals, or at least only causing very little unwanted effects in mammals, including man, whose genome does not comprise any copy of a thyx gene.

Method for Screening THYX Polypeptide Inhibiting Compounds

Another object of the invention is also the use of a THYX polypeptide such as defined in the description in a method for screening anti-bacterial or anti-viral compounds.

According to a first embodiment of a method for screening an anti-bacterial or anti-viral compound according to the invention a THYX polypeptide such as defined in the description could be used for screening molecules being attached thereon.

The attachment of the polypeptide to the molecule or substance can or not inhibit (antagonist molecule) the thymidylate synthase activity of said polypeptide.

Such molecules able to attach themselves to any of the polypeptides according to the invention comprise antibodies, oligonucleotides, other proteins and generally speaking, small molecules of any nature.

In such a screening test, the attachment of the candidate molecule to the polypeptides could simply be shown, one of both partners being labeled with a detectable compound (polypeptide of interest or candidate molecule), wherein the THYX polypeptide/candidate molecule complex is then visualized through the detection of the detectable marker, after removal of the non specifically linked candidate molecules.

By way of example, a screening test of a candidate molecule able to attach itself to a polypeptide according to the invention could advantageously comprise a first step where the polypeptide of interest or the candidate molecule is immobilized on a substrate, a second step where the second partner (candidate molecule or polypeptide of interest) is brought in the presence of the first compound previously immobilized on the substrate, a third step where one or more cleaning operations are performed in conditions appropriate for removing compounds being not specifically linked, and finally a fourth step where the complex optionally formed between the polypeptide of interest and the candidate molecule is detected.

In the embodiment of the screening test where the candidate molecule is previously immobilized on a substrate and subsequently brought in the presence of the polypeptide of interest according to the invention, the detection of the complex formed by the candidate molecule and the polypeptide of interest according to the invention could be advantageously preformed using an antibody such as described hereinabove.

In another embodiment of the screening test where the polypeptide of interest according to the invention is previously immobilized on a substrate, the candidate molecule will be advantageously labeled using a detectable marker prior to its contact with the immobilized polypeptide of interest.

Such a detectable marker could be radioactive or non radioactive, for example, fluorescent or could correspond to a ligand for a third partner used for detection like a biotin molecule.

Consequently, another object of the invention is also a method for screening a candidate molecule or substance interacting with a polypeptide according to the invention, said method comprising the steps of:

a) contacting a polypeptide in accordance with the invention with the candidate substance or molecule to be tested;

b) detecting the complexes optionally formed between said polypeptide and said candidate substance or molecule.

The invention is also relative to a set or a kit for screening a candidate molecule or substance interacting with a polypeptide according to the invention, said set comprising:

a) a polypeptide in accordance with the invention;

b) if need be, means required for detecting the complex being formed between said polypeptide and the candidate molecule or substance.

Method for Screening Anti-bacterial or Anti-viral Compounds in an Acellular System

According to such other method for screening anti-bacterial or anti-viral compounds according to the invention, the test for inhibiting the thymidylate synthase activity of a THYX polypeptide is carried out in a cellular system, for example in a cell culture lysate expressing the THYX polypeptide and which does not simultaneously express any polypeptide coded by a thyA gene.

The cells from which the cell lysate is obtained are preferably cells having the same features as those implemented in the method for screening in an acellular system being described more in detail earlier in the specification.

In short, the cells from which the cell lysate is obtained are respectively:

either cells naturally expressing THYX in the absence of THYA;

or cells in the genome of which the thyA gene has been inactivated and which are transfected with a recombinant vector expressing a thyx gene.

The cell lysate could be obtained from a culture of the cells as described hereinabove, for example, through sonication or by osmotic shock, according to techniques well known to those skilled in the art.

After the cell lysis step as such, the cell fragments could be removed in a centrifugation step at the end of which such cell fragments are to be found in the pellet, the centrifugation supernatant comprising, amongst others, all the proteins including the THYX protein, and wich is recovered for implementing the screening method.

According to the screening method in an acellular system, the thymidylate synthase activity is quantified respectively in control samples only containing the cell lysate and in test samples containing a candidate inhibiting compound, if need be, for a plurality of increasing concentrations of the candidate inhibiting compound.

Preferably, a plurality of test samples will be implemented, comprising a given candidate inhibiting compound, at increasing concentrations.

The thymidylate synthase activity could be quantified, more particularly, through detecting the oxidation of methylene-tetrahydrofolate, as described in the examples.

Still another object of the invention is a method for screening an anti-bacterial or anti-viral compound in vitro in an acellular system, characterized in that said method comprises the steps of:

a) preparing a cell lysate from a culture of cells expressing a THYX polypeptide in the absence of a polypeptide coded by a thyA gene;

b) adding to the cell lysate obtained in step a) the inhibiting compound to be tested;

c) comparing the thymidylate synthase activity respectively in the cell lysate as obtained in step a) and in the cell lysate as obtained in step b); and

d) selecting the candidate compounds for which some inhibition of the thymidylate synthase activity has been detected.

A further object of the invention is also a kit or a set for screening a thymidylate synthase inhibiting compound characterized in that it comprises:

a) a composition comprising a THYX polypeptide in solution or in lyophilized form;

b) optionally one or more reagents required for quantifying the thymidylate synthase activity.

According to a first embodiment, the composition containing the THYX polypeptide comprises a cell lysate prepared as described hereinabove.

According to a second embodiment, the composition comprising the THYX polypeptide comprises an amount of the THYX polypeptide in purified form adapted for the obtention of an assay sample in solution comprising a concentration of the THYX polypeptide ranging from 10⁻¹⁰ to 10⁻²M, preferably from 10⁻⁸ to 10⁻³M and most preferably, from 10⁻⁷M to 10⁻⁵M.

Method for Screening Anti-bacterial or Anti-viral Compounds in a Cell System

The invention also relates to using a nucleic acid coding a THYX polypeptide such as defined in the description in a method for screening anti-bacterial or anti-viral compounds.

Preferably the nucleic acid codes a THYX polypeptide selected amongst polypeptides comprising the amino acid sequences SEQ ID N^{o}1 to SEQ ID N^{o}37.

Most preferably, the nucleic acid is selected amongst the nucleic acids comprising the nucleotidic sequences SEQ ID N^{o}44 to SEQ ID N^{o}64.

The invention is also relative to a kit or a set for screening an anti-bacterial or anti-viral compound, characterized in that it comprises:

a) a recombinant expression vector comprising a nucleic acid coding a THYX polypeptide such as defined in the present description, under the control of a functional promoter in a host cell wherein its expression is being sought or in a host cell transfected with such a recombinant vector;

b) optionally one or more reagents required for quantifying the thymidylate synthase activity.

According to a second embodiment of a method for screening anti-bacterial or anti-viral compounds according to the invention, the activity of the thimydylate synthase activity inhibiting compounds of THYX could be tested in cell cultures expressing THYX, in the absence of thyA expression.

According to a first aspect, such a screening method could be implemented on cell cultures for the genome having a copy of the THYX gene, but no thyA gene, as for example Campylobacter jejune, Helicobacter pylori, Rickettsia prowazekii, Borrelia burgdorferi or Chlamydia cultures.

According to a second aspect, such a method for screening anti-bacterial or anti-viral compounds in a cell system could be implemented using cell cultures wherein the thyA gene has been inactivated and which have been transfected by a nucleic acid or a recombinant vector expressing a thyX gene in such cells, as for example the E.coli n^{o}X 2913 (ΔthyA) strain which has been transfected with an expression vector coding a THYX polypeptide.

Still another object of the invention is a method for screening an anti-bacterial or anti-viral compound characterized in that it comprises the steps of:

a) cultivating cells expressing the thyX gene in the absence of expression of the thyA gene in an appropriate culture medium;

b) contacting the cells with a candidate compound to be tested; and

c) selecting the candidate compounds inhibiting the thimydylate synthase activity of the polypeptide coded by the thyX gene.

The compounds selected by means of the above-mentioned screening method are those for which some inhibition of the thymidylate synthase activity is observed in the cells, as opposed to the thymidylate synthase activity as observed in the control cell cultures which are not put in the presence of candidate compounds to be tested.

The quantification of the thymidylate synthase activity could be achieved for example by incorporating a radiolabelled uracil in the DNA of the cultured cells, using techniques well known to those skilled in the art, the amount of radiolabelled dTTP in the DNA reflecting the thymidylate synthase activity level in the cell culture. In such a case, the cells as cultivated in step a) of the method are incubated in the presence of a radiolabelled uracil, for example (³H)-uracil or (¹⁴C)-uracil.

The radioactivity of dTTP in the DNA is measured after hydrolysis of DNA purified using standard techniques (radioactivity counter).

After lysis of the cells, for example, through sonication or osmotic shock, the cell lysates are filtered on a nitrocellulose membrane retaining the DNA, and thereafter the radioactivity contained on the filter is measured using an adapted radioactivity counter.

In the embodiment of the above-mentioned screening method, where the cultivated cells consist in cells having their genome which does not comprise any active copy of a thyA gene and which have been transfected with a recombinant vector comprising a DNA insert coding a THYX polypeptide, the recombinant vector will be selected so as to allow for the expression of the THYX polypeptide in the host cell being cultivated during the method.

Examples of recombinant vectors useful for implementing the above-described screening method are detailed hereinafter.

Recombinant Vectors able to be Used According to the Invention

The invention also relates to the use of a recombinant vector comprising a nucleic acid coding a THYX polypeptide such as defined hereinabove or a variant of such a polypeptide.

Advantageously, such a recombinant vector will comprise a nucleic acid selected amongst the following nucleic acids:

a) a nucleic acid coding a polypeptide having an amino acid sequence selected from the group of sequences SEQ ID N^{o}1 to SEQ ID N^{o}37 or a variant of such a polypeptide, optionally merged with a heterologous polypeptide;

b) a nucleic acid comprising a polynucleotide selected amongst sequences SEQ ID N^{o}44 to SEQ ID N^{o}64, or a variant of the latter;

c) a nucleic acid coding a THYX polypeptide having at least 95% amino acid identity with a polypeptide selected amongst the group consisting of the sequences SEQ ID N^{o}1 to SEQ ID N^{o}37 or a variant of the latter;

d) a nucleic acid having at least 95% nucleotide identity with a nucleic acid selected from the group consisting of the sequences SEQ ID N^{o}44 to SEQ ID N^{o}64 or a variant of the latter.

It is meant by “vector” herein a circular or linear DNA or RNA molecule being undiscriminately in the form of a single strand or a double strand.

Are preferred the expression vectors comprising, beside a nucleic acid coding a THYX polypeptide in accordance to the invention, regulatory sequences making it possible to direct the transcription and/or the translation thereof.

According to an advantageous embodiment, a recombinant vector according to the invention will more particularly comprise the following elements:

(1) regulatory elements for the expression of the nucleic acid to be inserted, such as promoters and enhancers;

(2) the coding sequence comprised in the nucleic acid in accordance to the invention to be inserted into such a vector, said coding sequence being arranged in phase with the regulatory signals described in (1); and

(3) appropriate transcription initiation and stop sequences.

Moreover, the recombinant vectors according to the invention could comprise one or more replication origins in the cell hosts wherein their expression is being sought, one or more selection markers.

By way of examples, the bacterial promoters could be the Lacl, LacZ promoters, the promoters of the RNA polymerase of the T3 or T7 bacteriophage, the PR or PL promoters for the lambda phage.

The eukaryote cell promoters will comprise the promoter of the thymidine kinase of the HSV virus or also the promoter of the mouse's metallothioneine-L.

Generally, for selecting an adapted promoter, those skilled in the art could advantageously refer to the above-mentioned work by SAMBROOK et al. (1989) as well as to the techniques as disclosed by FULLER et al. (1996).

Vectors particularly adapted for an expression of the nucleic acids according to the invention in bacteria are, for example, the pQE70, pQE60 or pQE-9 vectors (commercialized by QIAGEN company), the pBluescript, Page script, pNH8A; pNH16a, pNH18a, pNH46A vectors (commercialized by Stratagene corporation), the pKK223-3, pKK233-3, pDR540 and pRIT5 vectors (commercialized by Pharmacia corporation).

Vectors being particularly adapted for an expression in eukaryote cells are for example the pWLNEO, pSV2CAT, pOG44, pXT1 and pSG vectors (commercialized by Stratagene corporation), the pSVK3, pBPV, pMSG and pSVL vectors (commercialized by Pharmacia corporation).

A first vector preferably implemented within the scope of the invention is the pcDNA3 vector commercialized by Invitrogen corporation.

A second particularly preferred vector is the pBluescript SK (−) vector commercialized by Stratagene corporation.

The preferred bacterial vectors according to the invention are for example the pBR322(ATCC37017) vectors as well as vectors such as pAA223-3 (Pharmacia, Uppsala, Sweden), and pGEM1 (Promega Biotech, Madison, Wis., USA).

Are also to be mentioned other commercialized vectors such as the psiX174, pBluescript SA, pNH8A, pNH16A, pNH18A, pNH46A, pWLNEO, pSV2CAT, pOG44, pXTI, pSG(Stratagene) vectors.

They could also be vectors of the baculovirus type such as the pVL1392/1393 vector (Pharmingen) used for transfecting the cells of the Sf9 line (ATCC N^{o}CRL 1711) derived from Spodoptera frugiperda.

They could also be adenoviral vectors such as human adenovirus of 2 or 5 type.

A recombinant vector according to the invention could also be a retroviral vector as well as an adeno-associated vector (AAV). Such adeno-associated vectors are for example disclosed by FLOTTE et al. (1992), SAMULSKI et al. (1989), as well as McLAUGHLIN BA et al. (1996).

Most preferably, the pBAD TOPO vector commercialized by Invitrogen Corporation is implemented to allow for an expression of the thyX gene in E. Coli which is precisely regulated by the presence or the absence of arabinose in the culture medium of the transfected cells with such a recombinant vector, because the pBAD TOPO vector comprises the P_{BAD} promoter being arabinose inducible.

Examples of Thymidylate Synthase Activity Inhibiting Compounds of a THYX Polypeptide

Such thymidylate synthase activity inhibiting compounds of a THYX polypeptide are potentially anti-bacterial and/or anti-viral compounds without unwanted effects or with reduced unwanted effects for mammals, including man.

The growth of the transformants containing thyX in the E.coli X2913(ΔthyA) strain could be inhibited by trimethoprim, which is a specific inhibitor for the dihydrofolate reductase. This fact indicates that the growth depends on the folates.

Similarly, it is shown according to the invention that the thymidylate synthase activity of the polypeptide coded by the THYX gene of Pyrococcus abyssi is inhibited by a metabolic derivate of 5-fluorouracil, suggesting that the THYX polypeptide interacts with the fluoro-dUMP compound.

Other compounds able to inhibit the thymidylate synthase activity of a THYX polypeptide are respectively the antisense nucleic acids specifically hybridizing with the messenger RNA coding a THYX polypeptide and the antibodies raised against a THYX polypeptide.

Antisense Nucleic Acids

In order to inhibit or to block the expression of a nucleic acid coding a THYX polypeptide, those skilled in the art could use antisense polynucleotides.

Thus, the invention also relates to using an antisense polynucleotide or oligonucleotide able to specifically hybridize itself with the messenger RNA coding a THYX polypeptide and able to inhibit or to block the transcription and/or the translation thereof. Such a polynucleotide has the general structure being defined in the present description for the probes and the primers according to the invention.

Preferably, an antisense polynucleotide capable to be used according to the invention comprises a sequence corresponding to a sequence located in the region of the 5′ end of the messenger RNA, and most preferably in the vicinity of the initiation codon of the translation (ATG) of the nucleic acid coding the THYX polypeptide.

According to a second preferred embodiment, an antisense polynucleotide according to the invention comprises a sequence corresponding to one of the sequences located at the level of the exon/intron junctions of a gene coding the THYX polypeptide and most preferably, sequences corresponding to a splicing site.

An antisense polynucleotide according to the invention could be prepared from a nucleic acid coding a THYX polypeptide selected amongst the polypeptides with sequences SEQ ID N^{o}1 to SEQ ID N^{o}37.

An antisense polynucleotide according to the invention could be prepared from a nucleic acid selected amongst the nucleotidic sequences SEQ ID N^{o}44 to SEQ ID N^{o}64.

Generally, the antisense polynucleotides should have a length and a melting temperature suffificient for allowing to form an intracellular duplex hybrid with a sufficient stability for inhibiting the expression of the mRNA coding the subject THYX polypeptide. Strategies for building up antisense polynucleotides are more particularly disclosed by Green et al. (1986) and Izant and Weintraub (1984).

Methods for building antisense polynucleotides are also disclosed by Rossi et al. (1991) as well as in the PCT Applications WO 94/23026, WO 95/04141, WO 9218522 and in the European Patent Application EP 0 572 287.

Advantageously, an antisense polynucleotide according to the invention is 15 to 200 nucleotides long. A sense polynucleotide of the invention has therefore a length varying from 15, 20, 25, 30, 35, 40, 45 or 50 to 75, 100, 150 or 200 nucleotides.

In order to inhibit or to block the expression of a nucleic acid coding a THYX polypeptide such as defined in the description, one could also simultaneously use a plurality of antisense polynucleotides such as defined hereinabove, each of the antisense polynucleotides hybridizing with a distinct region of the gene or its messenger RNA.

Other methods for implementing antisense polynucleotides are for example disclosed by Sczakiel et al. (1995) or also disclosed in the PCT Application WO 95/24223.

Antibodies

THYX polypeptides such as defined according to the invention, more particularly polypeptides with amino acid sequences SEQ ID N^{o}1 to SEQ ID N^{o}37, or variants thereof as well as the homologous peptides, could be used for preparing antibodies which are able to be selected for the ability to inhibit or to block their thymidylate synthase activity.

Still another object of the invention is the use of antibodies raised against a THYX polypeptide for inhibiting or blocking the thymidylate synthase activity of such a polypeptide.

Such antibodies specifically raised against a THYX polypeptide represent a new illustrative example of a thymidylate synthase activity inhibiting compound of a THYX polypeptide according to the invention.

Such antibodies potentially represent anti-bacterial or anti-viral compounds.

It is meant herein by “antibody”, more particularly polyclonal or monoclonal antibodies or fragments thereof (for example F (ab)′₂, Fab fragments) or also any polypeptide comprising a domain of the initial antibody recognising the target polypeptide or polypeptide fragment according to the invention.

Monoclonal antibodies could be prepared from hybridomas according to the technique disclosed by KOHLER and MILSTEIN (1975).

The present invention is also relative to antibodies raised against a polypeptide such as described hereinabove or a fragment or a variant thereof, such as produced in the trioma technique as well as in the hybridoma technique as disclosed by KOZBOR et al. (1983).

The invention is also relative to single chain antibody fragments Fv (ScFv) such as disclosed in the U.S. Pat. N^{o} 4,946,778 or also by MARTINEAU et al. (1998).

The antibodies according to the invention also comprise antibody fragments obtained using phage banks from RIDDER et al., (1995) or also humanized antibodies (REIMANN et al., 1997; LEGER et al., 1997).

Pharmaceutical Compositions

Another object of the invention is also an anti-bacterial or anti-viral pharmaceutical composition comprising, as an active principle an antisense oligonucleotide specically hybridizing with a messenger RNA coding a THYX polypeptide such as defined in the present description, in association with one or more physiologically compatible excipients.

It is also relative to using an antisense oligonucleotide specifically hybridizing with the messenger RNA coding a THYX polypeptide such as defined in the present description for producing an anti-bacterial or an anti-viral drug.

Such a pharmaceutical composition will preferably comprise antisense oligonucleotide concentrations being at least equimolar with those of the corresponding messenger RNA in the cell.

Amongst the excipients useful in association with an antisense oligonucleotide such as defined hereinabove, are to be mentioned the synthetic cationic molecules binding to the anionic sites of the antisense oligonucleotide, which aids to the passage of the antisense oligonucleotide through the cell membrane via a non specific endocytosis, including those disclosed by Schofield in 1995 or those disclosed by BEHR in 1994.

Another useful excipient in association with an antisense oligonucleotide according to the invention is the Lipofectin™ compound, which comprises a 1:1 formulation of the quaternary ammonium compound DOTMA and dioleoylphosphatidylethalolamine, sonicated in the form of small unilamellar vesicles in water.

Another object of the invention is also an anti-bacterial or an anti-viral pharmaceutical composition comprising as an active principle, an antibody specifically raised against a THYX polypeptide such as defined in the description, in association with one or more physiologically compatible excipients.

It is also relative to using an antibody specifically raised against a THYX polypeptide such as defined in the description for producing an anti-bacterial or an anti-viral drug.

The invention is also relative to a method for preventing or for treating a bacterial or a viral disease, said method comprising a step for administrating a therapeutically efficient amount of an antisense oligonucleotide or antibody specific for a THYX polypeptide such as defined hereinabove.

A pharmaceutical composition according to the invention could be administered by any route, for example through intraveinous, intramuscular, oral or mucosal route, in association with a physiologically compatible carrier and/or adjuvant or excipient.

An antibody specifically raised against a THYX polypeptide is present in a pharmaceutical composition according to the invention in amounts adapted for a daily administration of 10 nanogrammes to 10 mg of antibody, preferably from 100 nanogrammes to 1 mg and more preferably from 1 pg to 100 pg antibody.

Techniques for formulating and administrating thymidylate synthase inhibiting compounds of a THYX polypeptide could be found by those skilled in the art in the following work: <<REMINGTON'S PHARMACEUTICAL SCIENCES-MACK publication co., Easton, Pa.>>, in its latest edition.

Use of Nucleotidic Probes and Primers Hybridizing with a Nucleic Acid Coding a THXY Polypeptide

As already set forth previously, the thyX genes were found according to the invention in various bacteria and viruses pathogenic in mammals, in particular bacteria pathogenic in man, and viruses.

Consequently, probes or primers derived from genomic nucleic acids or from the messenger RNA coding a THYX polypeptide are means for detecting the presence of a pathogenic bacterium or virus in a sample, and more specifically a biological sample taken from man or from an animal, for example a sample of saliva, tears, blood, plasma or serum or also from a biopsy sample or a smear.

The nucleic acids derived from any of the nucleotidic sequences coding a THYX polypeptide such as defined in the description, more particularly the nucleic acids with sequences SEQ ID N^{o}44 to SEQ ID N^{o}64 are useful for detecting the presence of at least one copy of a nucleotidic sequence selected amongst the sequences SEQ ID N^{o}44 to SEQ ID N^{o}64 or also of a fragment or a variant thereof in a sample.

Preferably, nucleotidic probes or primers according to the invention will have a length of 10, 12, 15, 18 or 20 to 25, 35, 40, 50, 70, 80, 100, 200, 500, 1000, 1500 consecutive nucleotides of a nucleic acid coding a THYX polypeptide or of a nucleic acid with a complementary sequence.

Alternately, a nucleotidic probe or primer according to the invention will consist in and/or comprise fragments with a length of 12, 15, 18, 20, 25, 35, 40, 50, 100, 200, 500, 1000, 1500 consecutive nucleotides of a nucleic acid coding a THYX polypeptide according to the invention, more particularly a nucleic acid selected amongst the sequences SEQ ID N^{o}44 to SEQ ID N^{o}64, or a nucleic acid with a complementary sequence.

The definition of a nucleotidic probe and primer according to the invention encompasses oligonucleotides hybridizing, in the strongly stringent hybridization conditions as defined hereinafter, with a nucleic acid coding a THYX polypeptide, in particular a nucleic acid selected amongst the sequences SEQ ID N^{o}44 to SEQ ID N^{o}64 or with a complementary sequence thereof.

Definition of the Hybridization Conditions

It is meant by strongly stringent hybridization conditions, as used herein, the following hybridization conditions.

Prehybridization

Same conditions as for the hybridization

duration: 1 night.

Hybridization

5×SSPE (0.9 M NaCl, 50 mM sodium phosphate pH 7.7, 5 mM EDTA)

5×Denhardt's (0.2% PVP, 0.2% Ficoll, 0.2% SAB)

100 μg/ml DNA of salmon's sperm

0.1% SDS

duration: 1 night.

Washing Operations

2×SSC, 0.1% SDS 10 min 65° C.

1×SSC, 0.1% SDS 10 min 65° C.

0.5×SSC, 0.1% SDS 10 min 65° C.

0.1×SSC, 0.1% SDS 10 min 65° C.

The parameters defining the stringency conditions depend on the temperature at which 50% of the coupled strands are separated from each other (Tm).

For the sequences comprising more than 360 bases, Tm is defined by the relationship:

Tm=81.5+0.41 (% G+C)+16.6 Log(cation concentration)−0.63 (% formamide)−(600/number of bases) (SAMBROOK et al., (1989), pages 9.54-9.62).

For sequences with a length lower than 30 bases, Tm is defined by the relationship: Tm=4(G+C)+2(A+T).

Under the appropriate stringency conditions, where the aspecific sequences do not hybridize, the hybridization temperature is approximatively from 5 to 30° C., preferably from 5 to 10° C. below Tm.

The above described hybridization conditions are implemented for hybridizing a nucleic acid being 200 base long and could be adapted depending on the length of the nucleic acid the hybridization of which is desired, or of the selected marking type, according to the techniques known to those skilled in the art.

The appropriate hybridization conditions could for example be adapted according to the teaching from the work by HAMES and HIGGINS (1985) or also from the work of AUSUBEL et al. (1989).

More particularly, it is to be noted that the hybridization level and specificity depend on various parameters such as:

a) the purity of the preparation of the nucleic acid on which the probe or the primer has to hybridize;

b) the base composition of the probe or of the primer, the G-C base pairs having a higher thermal stability than the A-T or A-U base pairs;

c) the length of the homologous base sequence between the probe or the primer and the nucleic acid;

d) the ionic strength: the hybridization rate increases with the increase of the ionic strength and the incubation time duration;

e) the incubation temperature;

f) the concentration of the nucleic acid on which the probe or the primer has to hybridize;

g) the presence of denaturants, such as agents promoting the break of hydrogene links, such as formamide or urea, increasing the stringency of the hybridization;

h) the incubation time, the incubation rate increasing with the incubation duration;

i) the presence of volume excluding agents, such as dextran or dextran sulfate, increasing the hybridization rate as they increase the effective concentrations of the probe and the primer and of the nucleic acid that should hybridize to, within the preparation.

A nucleotidic primer or probe according to the invention could be prepared using any adapted method well known to those skilled in the art, including through cloning and action of restriction enzymes or also through direct chemical synthesis according to techniques such as the phosphodiester method by NARANG et al. (1979) or by BROWN et al. (1979), the diethylphosphoramidite method by BEAUCAGE et al. (1980) or also the solid substrate technique as disclosed in the EU Patent EP 0,707,592.

Each of the nucleic acids according to the invention, including the above described oligonucleotidic probes and primers, could be labeled, if desired, by incorporating a marker being detectable by spectroscopic, photochemical, biochemical, immunochemical or also chemical means.

For example, such markers could comprise radioactive isotopes (³²p, ³³p, ³H, ³⁵S), fluorescent molecules (5-bromodeoxyuridin, fluorescein, acetylaminofluorene, digoxigenin) or also ligands such as biotin.

Probe labeling preferably occurs by incorporating labeled molecules within polynucleotides through primer extension, or also through addition on the 5′ or 3′ ends.

The oligonucleotide probes according to the invention could be used, more particularly, in hybridizations of the Southern type with genomic DNA or also in hybridizations with the corresponding messenger RNA when the expression of the corresponding transcript is sought for in a sample.

The probes according to the invention could also be used for detecting PCR amplification products or also for detecting mismatch pairings.

Nucleotidic probes and primers according to the invention could be immobilized on a solid substrate. Such solid substrates are well known to those skilled in the art and comprise surfaces of microtitration plate wells, polystyrene beds, magnetic beds, nitrocellulose bands or also microparticles such as latex particles.

Consequently, another object of the invention is the use of a nucleic probe or primer hybridizing with a nucleic acid coding a THYX polypeptide, such as defined in the description, in a method for detecting a bacterium or a virus, more particularly a bacterium or virus pathogenic in mammals, including man.

The present invention also relates to a method for detecting the presence of a nucleic acid such as described hereinabove in a sample, said method comprising the steps of:

1) contacting one or more nucleotidic probes according to the invention with the sample to be tested;

2) detecting the complex optionally formed between the probe(s) and the nucleic acid present in the sample.

According to a particular embodiment of the detection method according to the invention, the oligonucleotidic probe(s) is/are immobilized on a substrate.

According to another aspect, the oligonucleotidic probes comprise a detectable marker.

The invention additionally relates to a set or a kit for detecting the presence of a nucleic acid according to the invention in a sample, said set comprising:

a) one or more nucleotidic probes such as described hereinuder;

b) if need be, the reagents required for the hybridization reaction.

According to a first aspect, the detection set or kit is characterized in that the probe(s) is/are immobilized on a substrate.

According to a second aspect, the detection set or kit is characterized in that the oligonucleotidic probes comprise a detectable marker.

According to a particular embodiment of the above described detection kit, such a kit will comprise a plurality of oligonucleotidic probes in accordance with the invention able to be used for detecting target sequences of interest or alternatively for detecting mutations in the coding regions or the non coding regions of the nucleic acids according to the invention, more particularly nucleic acids with sequences SEQ ID N^{o}44 to SEQ ID N^{o}64 or nucleic acids with complementary sequence.

Thus, the probes according to the invention immobilized on a substrate could be ordered in matrices such as the “DNA chips”. Such ordered matrices have been more particularly described in the U.S. Pat. N^{o} 5,143,854, in the PCT Applications N^{o} WO 90/150 70 and 92/10092.

Substrate matrices on which oligonucleotidic probes have been immobilized at a high density are for example disclosed in the U.S. Pat. N^{o} 5,412,087 and in the PCT Application WO 95/11995.

The nucleotidic primers according to the invention could be used for amplifying any one of the nucleic acids according to the invention, and more particularly, all or part of a nucleic acid with sequences SEQ ID N^{o}44 to SEQ ID N^{o}64, or also a variant thereof.

Another object of the invention relates to a method for amplifying a nucleic acid according to the invention, and more particularly a nucleic acid with sequences SEQ ID N^{o}44 to SEQ ID N^{o}64 or a fragment or a variant thereof contained in a sample, said method comprising the steps of:

a) contacting the sample wherein the presence of the target nucleic acid is suspected with a pair of nucleotidic primers the hybridization position of which is located respectively on 5′ side and on 3′ side of the region of the target nucleic acid the amplification of which is being sought for, in the presence of the reagents required for the amplification reaction; and

b) detecting amplified nucleic acids.

In order to implement the above defined amplification method, one should advantageously use any of the hereinabove described nucleotidic primers.

Yet another object of the invention is a set or a kit for amplifying a nucleic acid according to the invention, and more particularly all or part of a nucleic acid with sequences SEQ ID N^{o}44 to SEQ ID N^{o}64, said set or kit comprising:

a) a nucleotidic primer couple in accordance with the invention, the hybridization position of which is located respectively on 5′ side and on 3′ side of the target nucleic acid the amplification of which is being sought;

b) if need be, the reagents required for the amplification reaction.

Such an amplification set or kit will advantageously comprise at least one pair of nucleotidic primers such as described hereinabove.

Use of a Nucleic Acid Coding a THXY Polypeptide as a Selection Marker.

There is a constant need in the state of the art for novel selection marker genes, particularly in methods aiming at introducing one or more genes of interest into a host organism, for example a host cell.

The selection marker genes, being carried by the DNA molecule coding the gene(s) of interest which are to be introduced into the host organism or in the host cell, and which are consequently introduced into the host cell simultaneously with the genes of interest, make it possible to select the recombinant host cells.

A nucleic acid coding a THYX polypeptide, when being used for transfecting auxotrophic host cells for the thymidine synthesis, is an excellent selection marker of transformation, transfection or recombination event of the host cell. Indeed, introducing a nucleic acid coding a THYX polypeptide simultaneously with one or more genes of interest into the host cell restores the autotrophy of the host cell being successfully subjected to the transfection, transformation or recombination.

In this way, one can select host cells having been subjected to the transfection, transformation or recombination event through medium selection pressure, and more particularly, cultivating the host cells in the absence of thymidine. In such a case, only the host cells having been recombined survive in the absence of thymidine in the culture medium.

Additionally, a nucleic acid coding a THYX polypeptide is totally non toxic for the environment.

Vectors comprising a nucleic acid coding a THYX polypeptide as a selection marker gene could be prepared from conventional vectors using techniques well known to those skilled in the art, for example from the preferred vectors according to the invention.

Still another object of the invention is the use of a nucleic acid coding a THYX polypeptide such as defined in the description as a selecting marker of a genetic transfection, transformation or recombination event of a host cell or a host organism.

It is also relative to a cloning and/or expression vector comprising a nucleic acid coding a THYX polypeptide such as defined in the description as selection marker genes for a genetic transfection, transformation or recombination event.

Reaction Media for the Thymidylate Synthase Activity of THXY and their Uses, more Particularly in Screening Methods

The inventors' work on the ThyX activity made it possible to identify the enzyme mechanism and thereby to optimize the conditions of the reaction by showing the importance of adding some compounds in the reaction medium.

Another aim of the invention is therefore the use of such media and the application thereof.

It also relates to a reaction medium for the thymidylate synthase activity of ThyX characterized in that it comprises reduced flavins and CH₂H₄folate.

More particularly, the reduced flavins of such a medium are obtained through in situ reduction of oxidized flavins. The oxidized flavin concentration is then 50 μM to 1 mM, preferably 0.5 mM. Preferably, the oxidized flavins are flavin mononucleotide (FMN) and/or flavin adenine dinucleotide (FAD). The flavin reduction could occur through chemical, enzymatic, photochemical or electrochemical route, and more particularly, with NADH (β-nicotinamide adenine dinucleotide) and/or NADPH (β-nicotinamide adenine dinucleotide phosphate).

Moreover, the CH₂H₄folate concentration is 50 μM to 2 mM, preferably 1 mM.

Preferably, the medium could additionally comprise dUMP (uridine 5′-monophosphate) at a concentration ranging from 1 μM to 800 μM, more preferably 500 μM.

When flavin reduction is performed with NADH and/or NADPH, the NADH concentration ranges from 0.1 to 1 mM, and the NADPH concentration from 0.5 to 5 mM.

The invention further aims at screening methods. In particular, an object of the invention is to provide a method for screening an anti-bacterial or an anti-viral compound in vitro in an acellular system characterized in that said method comprises the steps of:

a) preparing a cell lysate from a cell culture expressing a THYX polypeptide in the absence of a polypeptide coded by a thyA gene and comprising a medium according to the invention;

b) adding to the cell lysate obtained in step a) the inhibiting compound to be tested;

c) comparing the thymidylate synthase ThyX activity respectively in the cell lysate obtained in step a) and in the cell lysate obtained in step b); and

d) selecting the candidate compounds for which an inhibition of the thymidylate synthase ThyX activity has been detected.

In such a screening method, dTMP and ³H are preferably used as markers of the thymidylate synthase ThyX activity.

The invention also encompasses kits or sets for screening a thymidylate synthase ThyX inhibiting compound characterized in that it comprises:

a) a composition comprising a THYX polypeptide as well as a medium according to the invention, in solution or in freeze-dried form;

b) optionally, one or more reagents required for quantifying the thymidylate synthase ThyX activity. Alternatively, the kit or set for screening an anti-bacterial or an anti-viral compound, could also comprise:

a) a recombinant expression vector comprising a nucleic acid coding a THYX polypeptide under the control of a functional promoter in a host cell wherein its expression is being sought or a host cell transfected with such a recombinant vector;

b) a medium according to the invention;

c) optionally, one or more reagents required for quantifying the thymidylate synthase ThyX activity.

Other features and advantages of the invention are given by way of illustration in the following examples referring to the figures.

FIG. 5 illustrates biochemical analyses of the ThyX of H. Pylori.

(A) 12% SDS-PAGE and immublot analyses of ThyX isolated protein of H. pylori. 1.5 (Coomassie) and 0.3 (anti-V5) μg of pure protein have been detected respectively through coloration with Coomassie Blue or using monoclonal antibodies against an anti-V5 epitope (Invitrogen), through chemiluminescent detection. The expected molecular weight of ThyX of H. pylori is 31.5 kDa

(B) The spectroscopic analyses of ThyX of H. pylori indicate that ThyX is a flavoprotein. The 10 μM absolute spectrum of isolated enzyme (plain line) and of co-factor after its protein release (dashed line). The window shows the spectrum for the oxidized enzyme and the reduced dithionic protein.

(C) The release activity of the Tritium of the purified ThyX protein of H. pylon has been recorded in the presence (+MTHF) and in the absence (−MTHF) of CH₂H₄tetrahydrofolate.

FIG. 6 illustrates the dTMP formation activity of the ThyX protein of H. pylori. The enzyme reactions were performed as described in table 1, using marked dUMP in position 6. The reaction products were analyzed using a reverse phase column C 18 through isocratic elution, using 10 mM of phosphate buffer. The elution durations for dUMP and dTMP were determined using the genuine references.

The invention is further illustrated, without any limitation, by the following examples.

### EXAMPLES

### Example 1

Expression of THYX Polypeptide of Helicobacter Pylori in E. Coli

The DNAs comprising the open reading frame coding the THYX polypeptide of Helicobacter pylori (strain 26.695) (sequence SEQ ID n^{o}21) were obtained through PCR amplification using primers specific for sequences SEQ ID N^{o}38 and SEQ ID N^{o}39 from the GHPEH26 clone publicly available from the AMERICAN TYPE CULTURE COLLECTION under the access number n^{o}628.507.

The DNA comprising the open reading frame coding the THYX polypeptide of Pyrococcus. abyssi (strain ORSAY) (SEQ ID n^{o}12) was prepared through PCR amplification using primers specific for sequences SEQ ID N^{o}40 and SEQ ID N^{o}41 from the chromosomic DNA of H. pylori (HP 1533).

The DNA comprising the open reading frame coding the THYX polypeptide of Campylobacter jejuni (strain NCTC 11168) (SEQ ID N^{o}27) was prepared through PCR amplification using primers specific for sequences SEQ ID N^{o}42 and SEQ ID N^{o}43 from the genomic DNA publicly available from the ATCC (American Type Culture Collection) under the access number 7008199.

The PCR amplification products were cloned in the pBAD TOPO TA vector activated by topoisomerase-l, commercialized by Invitrogen corporation, allowing for a strictly regulated expression of the gene of interest artificially inserted into the vector, in the E. coli bacterium.

The E. coli clones were characterized through sequencing DNA inserts contained in the pBADTOPOTA vector.

The theoretical molecular weights of the THYX polypeptides of H. pylori (HP1533), P. abyssi (PAB 0861) and C. jejuni (NCTC 11168) including an amino-terminal translation activator and carboxy-terminal V5 epitopes and histidin, are respectively 31.5 kDa, 33.7 kDa and 28 kDA.

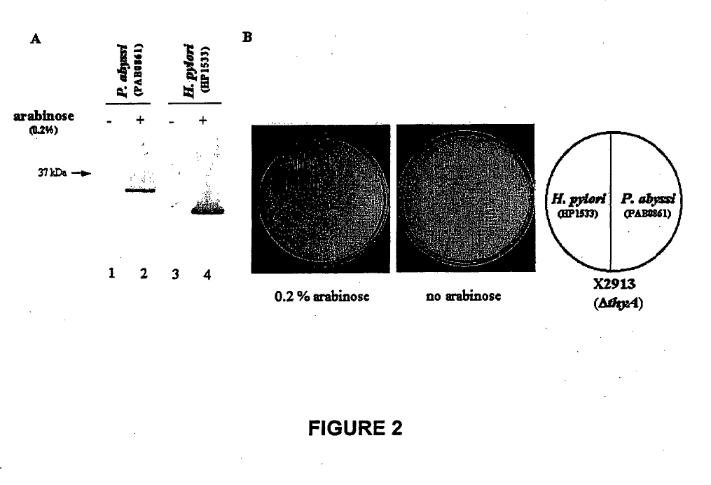

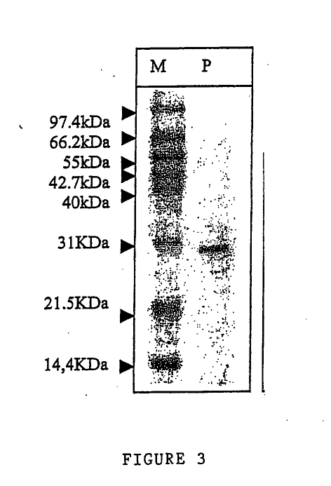

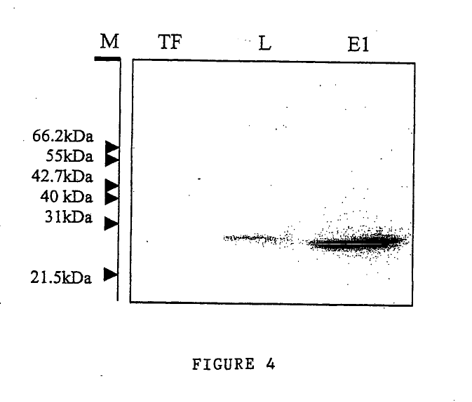

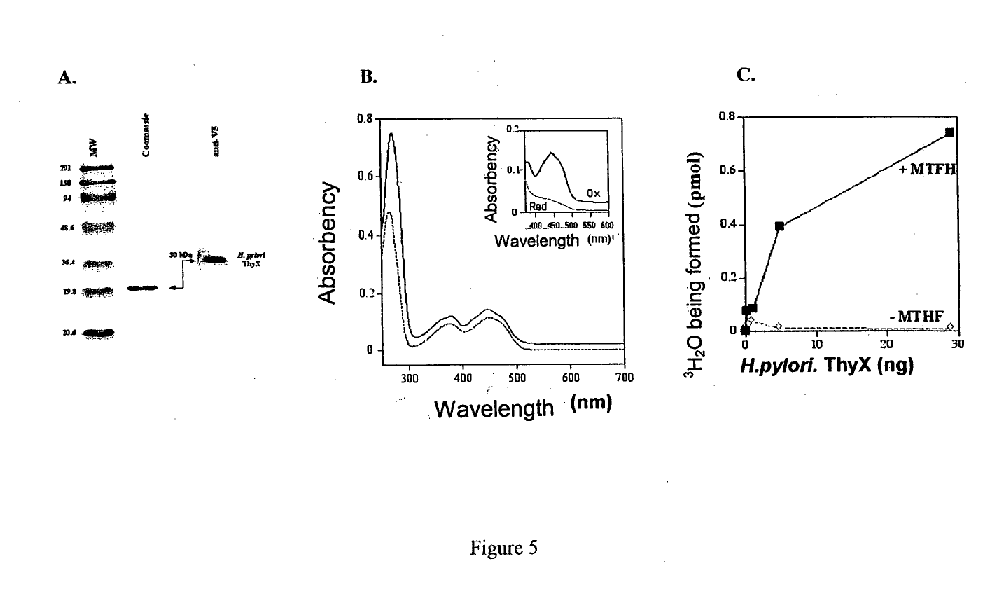

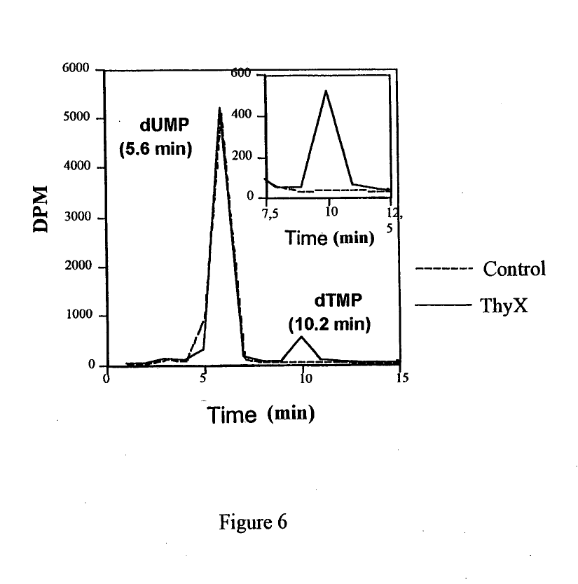

The results presented in FIG. 2A show that the expression of the THYX polypeptide is induced in the presence of 0.2% of Arabinose in the culture medium, as this was detected using anti-V5 monoclonal antibodies commercialized by Invitrogen Corporation and used according to the manufacturer's recommendations.

Then the ability of the THYX polypeptides of H.pylori and P.abyssi to allow for the growth of the E.coli X2913 (Δthya572) strain, auxotrophic for thymidine, to multiply in the absence of thymidine has been tested.

The growth ability of the E.coli strains expressing THYX polypeptides of H. pylori and P. abyssi was determined after culture of the recombinant E. coli cells for 3 to 4 days in the presence or in the absence of a 0.2% concentration of Arabinose on a thymidine free M9 minimum agar medium (Michaels et al., 1990).

The results are presented in FIG. 2B.

As can be seen from the results in FIG. 2B, the expression of the thyX gene of H. pylori in the E. coli Chi2193 strain wich was induced by Arabinose, made it possible to complement such an E. coli strain initially deficient in thymidylate synthase activity and to restore the prototrophy of such an E. coli strain for thymidine.

On the other hand, the thyx gene of P. abyssi, after induction by Arabinose, did not made it possible to complement the E. coli X 2193 strain, reflecting the inability of a hyperthermophilic protein to be functional in a mesophilic host.

Both recombinant E.coli strains were nevertheless able to multiply in a minimum agar medium in the presence of thymidine at the final concentration of 50 μg/ml.

The Helicobacter pylori GHPEH26 clone contains a DNA insert of 1.5421 kb corresponding to nucleotides 1613133-1611.613 of the ORF reference AE00511, locus 10, HP 1533, referenced in the data bases such as DNA seq. Acc: AE000 51l.

The GHPEH26 clone is commercialized by TIGR/ATCC Microbial Genome Special Collection Corporation. The thyX gene of H. pylori obtained through PCR from such a clone represents a 693 pb long DNA fragment.

The results of electrophoresis SDS PAGE gel illustrated in FIG. 3 show that the E. coli strain transformed with DNA of Campylobacterjejuni produces a THYX polypeptide with the expected molecular weight.

Additionally, the immuno-imprint results illustrated in FIG. 4 show that the THYX protein of Campylobacterjejuni could be efficiently purified on a Nickel Ni-NTA column.

The above described results represent the first experimental demonstration that the conversion reaction of dUMP into dTMP could be performed in the cell by a thymidylate synthase other than a thymidylate synthase coded by a thyA gene, i.e. by the thymidylate synthase coded by a thyx gene.

### Example 2

Identification of a THYX Polypeptide Family

Through the analysis of sequences of about 50,000 genes referenced in the data base of <<clusters>> of orthologous proteins (Tatuson et al., 2000) by a similarity research iterative method (Altschul et al., 1997), it was shown that the THYX sequences, similar to the THY1 sequence of H. pylori (HP 1533), were the only gene family having a mutually exclusive distribution with thyA, with the single exception of Mycobacterium tuberculosis which simultaneously comprises a thyX gene and a thyA gene.

The similarity iterative research was performed using the PSI-BLAST iterative program (Version 2.0) using various THYA sequences as <<lures>>. Selections (<<hits>>) having an expected value lower than 1.10⁻⁵ were considered as statistically significant. A threshold value for recruiting alignments in the successive iterations was 0.02.

The non-exhaustive results of the above mentioned protein similarity analysis are shown in Tables 1 and 2 hereinafter.

TABLE 1

Comparison

| | comparison | |
|---|---|---|
| | (%) | Similarity (%) |
| A. aeolicus | 26 | 47 |
| A. pernix | 25 | 38 |
| B. anthracis | 30 | 47 |
| B. burgdorferi | 23 | 38 |
| phi-C31 bacteriophage | 25 | 40 |
| B. lactis | 23 | 42 |
| C. difficile (partial) | 40 | 59 |
| C. diptheria | 25 | 45 |
| C. glutamicum | 23 | 42 |
| C. jejuni | 56 | 72 |
| Chlamydia sp. | 22 | 36 |
| Chlorella virus | 20 | 32 |
| D. discoideum | 23 | 41 |
| D. vulgaris | 23 | 46 |
| G. sulfuureducens | 28 | 48 |
| Gp16 (bacteriophage) | 24 | 39 |
| Gp48 (bacteriophage) | 25 | 44 |
| H. pylori (HP1533) | 100 | 100 |
| H. pylori (jhp1421) | 96 | 97 |
| H. salinarium | 22 | 43 |
| Halobacterium sp. NRC-1 | 22 | 43 |
| M. avium | 24 | 41 |
| M. bovis | 24 | 41 |
| M. leprae | 21 | 40 |
| M. tuberculosis | 23 | 39 |
| P. abyssi | 23 | 47 |
| P. furiosus | 27 | 52 |
| P. horikoshii | 25 | 46 |
| R. capsulatus | 18 | 38 |
| R. prowazekii | 22 | 41 |
| Roseophage S101 | 18 | 36 |
| S. coelicolor | 17 | 33 |
| S. solfaraticus | 26 | 45 |
| Synechocystis sp. | 19 | 34 |
| T. acidophilum | 21 | 42 |
| T. denticola | 28 | 48 |
| T. maritime | 28 | 46 |
| T. pallidum | 22 | 37 |
| T. volcanium | 20 | 34 |

%: The identity and similarity percentages as set forth in table 1 hereinabove were obtained after an iterative research using the BLAST or Psi-BLAST software, exclusively using the default parameters. The non-exhaustive results in Table 1 show that numerous bacteriophages, bacteria and viruses have a copy of a gene coding a THYX polypeptide.

TABLE 2

| Species | thyA¹ | thyX | DHFR | tdk | upp | Comments |
|---|---|---|---|---|---|---|
| Bacteria: | | | | | | |
| Campylobacter | − | + | − | − | + | Food poisonings |
| Jejuni | | | | | | |
| Helicobacter | − | + | − | − | − | Forming stomach |
| pylori | | | | | | ulcers |
| Rickettsia | − | + | − | − | − | Typhus causal |
| prowazekii | | | | | | agent |
| Borrelia | − | + | − | − | − | Involved in |
| burgdorferi | | | | | | Lyme's |
| | | | | | | disease |
| Treponema | − | + | − | − | + | Syphilis |
| pallidum | | | | | | causal agent |
| Chlamydia | − | + | + | − | + | Compulsory |
| (3 species) | | | | | | intracellular |
| | | | | | | pathogens |
| Mycobacterium | + | + | + | − | + | Tuberculosis |
| tuberculosis | | | | | | |
| Thermotoga | − | + | + | + | + | Thermophilic |
| maritima | | | | | | |
| Archaebacteria | | | | | | |
| Pyrococcus | − | + | − | − | + | Hyper- |
| abyssi | | | | | | thermophilic |
| Pyrococcus | − | + | − | − | − | Hyper- |
| horikoshii | | | | | | thermophilic |
| Eukaryotes: | | | | | | |
| Dictyostelium | N.A | + | N.A | N.A | N.A | No accessible |
| discoideum | | | | | | complete |
| | | | | | | sequence |
| | | | | | | genomic |
| Virus: | | | | | | |
| Bacterial and | − | + | N.A | N.A | N.A | |
| eukaryotic | | | | | | |
| DNA viruses | | | | | | |
| (5 species - | | | | | | |
| which ones?) | | | | | | |

¹thyA, gene coding the thymidylate synthase required for de novo synthesis of dTMP; thyX, new gene family involved in the biosynthesis of pyrimidines; DHFR, dihydrofolate reductase essential for recycling a methyl donor essential in the dTMP synthesis; tdkt thymidine kinase required for recovering the exogenous thymidine which is subsequently converted into dTMP; UPP, uracylphosphoribosyl transferase (UPRTase) required for recovering uracil.

In Table 2 hereinabove, the results are presented of the tests of the presence, more particularly of the thyX and thyA genes in some of the organisms as listed in Table 1.

The results set forth in Table 1 hereinabove show that the thyX genes are present both in bacteria, in bacteriophages and in viruses. Amongst the eukaryotes, the D.dyscoidum organism is the only organism carrying a copy of a thyX gene.

The single presence of a thyX gene in numerous bacteria and the eukaryotic DNA virus as well as in numerous bacteria pathogenic in man makes it a preferred target for anti-bacterial or anti-viral compounds which do not interfere with the metabolic route of the thymidylate synthase THYA present in man.

Additionally, amongst the organisms listed in Table 1 hereinabove many of them do not comprise DHFR genes coding a dihydrofolate reductase, which is required for recycling an essential cofactor of the metabolism of thymidylate, the CH₂H₄-folate.

### Example 3

Site Directed Mutagenesis of the THXY Gene from Helicobacter Pylori

A. Materials and Methods

For obtaining the mutants 1 to 6 as described in the <<Results>> Section, the site directed mutagenesis was performed using the <<Quick Change™>> kit commercialized by Stratagene Corporation in accordance with the manufacturer's recommendations.

For obtaining the mutants 7 and 8 as described in the <<Results>> Section, the site directed mutagenesis was performed using the <<QuickChange™ Multi Site-Directed Mutagenesis Kit>> kit commercialized by Stratagene Corporation in accordance with the manufacturer's recommendations.

The starting DNA (<<Template>>) being used is the pBAD TOPO plasmid, commercialized by InVitrogen Corporation, wherein there was inserted the thyX gene of Helicobacter pylori 26695.

The preserved serine 107 residue (<<AGT>> codon) was respectively replaced by cysteine (<<TGC>> codon) or alanine (<<GCT>> codon) residues using the appropriate mutagenic oligonucleotides.

The Tyrosine 110 residue (<<TAC>> codon) was respectively replaced by threonine (<<ACT>> codon) and phenylalanine (<<TTC>> codon) residues using the appropriate mutagenic oligonucleotides.

The Glutamate 142 residue (<<GAA>codon) was respectively replaced by Alanine (<<GCT>> codon) and Aspartate (<<GAT>> codon) residues using the appropriate mutagenic oligonucleotides.

The Histidine 71 residue (<<CAT>> codon) was replaced by the Glutamine residue (<<CAA>> codon) using the appropriate mutagenic oligonucleotides.

The Glutamate 205 residue (<<GAA>> codon) was replaced by the Leukine (<<TTA>> codon) residue using the appropriate mutagenic oligonucleotides.

B. Results

The mutagenesis experiments showed that the E. coli Chi2193 bacteria transformed by the pBAD TOTO TA vectors respectively containing DNA inserts coding the THYX protein wherein the preserved serine amino acid residue was respectively replaced by a cysteine or an alanine residue, expressed a mutated THYX protein containing a flavin.

On the other hand, both mutant transformed bacteria lost their ability to restore the prototrophy for the thymidine.

These results show that the preserved serine amino acid residue is essential for the catalytic activity of the THYX protein.

Additionally, the ability of the thyX gene to complement the bacteria for the thymidylate synthase activity is lost when the Tyrosine 110 and Glutamate 142 residues are mutated.

A mutation through substitution of Glutamine for Histidine 71 leads to the production of a polypeptide capable to interact with the flavin co-factor, but which does not complement the bacteria for the thymidylate synthase activity.

Substituting a Leukine residue for the Glutamate 205 residue blocks the production of the THYX polypeptide.

In additional experiments, the thyX gene of the Rhodobacter capsulatus bacterium was inactivated (through <<knock out>>). The bacteria lacking THYX protein expressed an auxotrophy phenotypic character for thymidine, i.e. they only multiply in the presence of thymidine. These latter results clearly show that the THYX proteins are essential for the thymidine metabolism. Such experiments show that the thyX proteins are essential to the bacterial growth in the absence of thymidine, showing the usefulness of the thyX gene or the THYX polypeptide as a therapeutic target.

### Example 5

Identification of the Reaction Mechanism of THXY

For identifying the biochemical reactions catalyzed by the ThyX proteins, a ThYX of H. pylori bearing a Histidine marker on its carboxy end was purified from acellular extracts from _{X}2913 strain of E. coli subjected to an induction by arabinose in affinity chromatography of immobilized nickel.

Open reading frames coding hypothetic proteins now found to correspond to the thyX genes of P. abyssi (PAB0861) and H. pylori (HP1533), were obtained through PCR, using specific primers and chromosomic DNA of P. abyssi and the GHPEH26 clone (American Type Culture Collection n^{o} 628507), respectively, as matrices. The PCR products were cloned in the pBAD TOPO® TA I-activated vector of topoisomerase (Invitrogen), allowing for the strictly controlled expression of the gene in E. coli. All the plasmidic clones were confirmed through DNA sequencing. The expected molecular masses of PAB0861 and HP1533, comprising an amino-terminal translation activating sequence and a carboxy-terminal V5 region and hexahistidin epitopes, are respectively 33.7 and 31.5 kDa. The expression of the protein was induced with 0.2% L-arabinose. The expressed proteins were detected through the use of V5-specific monoclonal antibodies (Invitrogen) in accordance with the manufacturer's recommendations.

The biologically active ThyX protein with a labeled antigenic site of H. pylori was purified from 200 ml of pGL2/E. coli X2913 [ΔthyA (table 3)] culture after 2 hours induction by 0.2% of L-arabinose. A QIA expression kit (Qiagen) under standard endogenous conditions was used for the purification as indicated by the manufacturer, comprising 10% (volume/volume) glycerol in all the buffers. The resulting protein samples were dialyzed against 50 mM of a phosphate, at pH 7.4 and 10% (volume/volume) glycerol buffer after elution so as to remove imidazole. The protein concentration in pure samples was evaluated through A₂₈₀ reading, justifying the A₂₈₀ absorbency of the flavin co-factor with a non covalent link, at 35560 M⁻¹ cm⁻¹, calculated with respect to the known amino acid sequence, and was used for the ThyX apoprotein of H. pylori.

The resulting protein preparations (purity>95%) usually contained 1 to 2 mg/ml of protein, with a molecular mass of approximately 31 kDa on SDS-PAGE gels (the ThyX expected molecular mass of H. pylori is 31.5 kDa) (FIG. 5A), and had a light yellow colour. The size exclusion chromatography with Superdex 200 using standard molecular weight markers, showed an endogenous molecular mass of 111 kDa (r=0.9874) for such a protein, suggesting that its active form could correspond to a homotetramer. Spectroscopic analyses of the isolated (oxidized) protein showed absorbency features typical for a flavoprotein (FIG. 5B), with large peaks at 447.5 and 375 nm. Such absorption peaks were found to be absent from the dithionite reduced enzyme. Similar absorption features were found for the co-factor after its release from the protein through denaturation using heat at 80° C. for 5 minutes. Using HPLC chromatography, the co-factor associated to the ThyX of H. pylori was identified as a FAD (flavin-adenine dinucleotide). It has been considered that the various enzyme preparations of ThyX of H. pylori contain 0.4 to 0.5 molecules of FAD per monomer. Globally, such spectroscopic properties indicate that the ThyX of H. pylori is a flavoprotein and/or uses flavin co-factors in the catalysis.

In dTMP formation, the loss of tritium from [5-H]dUMP in the solvent is a compulsory intermediary, allowing to quantify the thymidylate-synthetase activity after removal of radioactive nucleotides from the reaction mixtures (ROBERTS, 1966). In order to tackle the biochemical mechanism, wherein ThyX could circumvent the requirement for the ThyA in the de novo synthesis of thymydilate, the purified ThyX protein was used to the same extent.

### Example 6

Optimization of the ThyX Reaction Conditions

N⁵,N¹⁰—CH₂H₄folate was non-enzymatically formed, through incubation of 2 mM of tetrahydrofolic acid (Sigma®) with 100 mM of β-mercaptoethanol and 20 mM of formol for 30 minutes in the dark and at room temperature. The tritium release dosages with a purified enzyme were achieved in 50 mM of Tris-Cl, at pH 7.9, comprising a 1 mM CH₂H₄folate preparation obtained as described hereinabove. The control reactions were achieved under analogous conditions, without tetrahydrofolic acid. The reactions at 50 μl were started through the addition of 6 μM of [5-³H]dUMP, a specific activity at 16,2 Ci/mmol (Amersham) and stopped after 60 minutes at 37° C. by two extractions with 250 μl of active carbon [10% (weight/volume) of Norit A] in 2% trichloroacetic acid so as to remove the nucleotides from the reaction mixtures. The remanent radioactivity in the supernatant was determined according to MYLLYKALLIO (2000).

As can be seen from FIG. 5C, the ThyX catalyzes in vitro the tritium release from (5-³H)dUMP depending on the protein concentration and in a CH₂H₄folate dependent way, showing the biochemical activity of the ThyX proteins. Optimized reaction conditions are listed in Table 3.

TABLE 3
Optimization of reaction conditions for the ThyX protein of
H. pylori

| | nmol of released ³H/mg of |
|---|---|
| Test conditions | protein (60 minute incubation) |
| ThyX of H. pylori: | |
| Complete | 63.0 (100%) |
| Complete, −protein | 0.7 (1.1%) |
| Complete, −H₄folate | 0.8 (1.2%) |
| Complete, −NADH, −NADPH, −FMN | 2.2 (3.4%) |
| Complete, −FMN | 2.70 (4.3%) |
| Complete, −NADPH | 37.3 (59.2%) |
| Complete, −NADH | 19.0 (30.1%) |
| Complete, +500 μM Dump | 9.3 (14.8%) |
| Complete, +500 μM UMP | 64.4 (102.2%) |
| ¹The complete test contains 50 mM of Tris-HCl, pH 7.0, 1 mM of CH₂H₄folate preparation, 10 mM of MgCl₂, 2 mM of NADPH, 1 mM of NADH, 0.5 mM of FMN and 9 μM of 3H-dUMP (specific activity 1.7357 Ci/mmol). The CH₂H₄folate preparation is obtained through incubation for 30 minutes in the dark of 2 mM of H₄-folate, 96 mM of 2-mercaptoethanol and 42 mM of formaldehyde and 50 mM of Tris-HCl. In the reaction, FMN | |
| #could be efficiently replaced by FAD (flavin adenine dinucleotide). In such reaction conditions, 20 μM of dUMP and 100 μM of CH₂H₄folate are sufficient for saturating the release activity of tritium of the ThyX protein from H. pylori during 60 minutes incubation. | |

Surprisingly, it has been found that the addition of flavin reduced nucleotides drastically increases the tritium release activity of the ThyX from H. pylori (=0.01 μmol of H₂O formed per min and per mg of protein, as measured during additional experiments of absorption time).

Similarly, it has been shown by means of simple competition experiments that the ThyX activity does not use UMP as a substrate (Table 3) and that such an activity is inhibited by dTMP micromolecular concentrations. The tritium release activity of the ThyX from H. pylori is directly linked to the formation of dTMP (FIG. 6).

Experimental results therefore clearly showed that the ThyX proteins act as a dUMP dependent thymidilate-synthetase (FIGS. 5, 6).

Moreover, it has also been shown that the fluoro-dUMP acts as a THYX protein inhibitor. Such results show that the monophosphate deoxynucleotides could be used for identifying new THYX inhibitors.

For a practical application, those results also show that the tests are also useful for screening THYX proteins.

Consequently, while the catalysis through ThyX depends on the reduced flavin nucleotides, the ThyA uses H₄folate electrons for forming the methyl functional group. The CH₂H₄folate acts in the reaction catalyzed by ThyX only as a carbon donor, thereby leading to the formation of H₄folate as a reaction product. Such a reaction mechanism clearly explains the reason why dihydrofolate-reductase is not indispensable to the formation of thymidylate by the ThyX proteins (Table 4).

TABLE 4

| Species | thyA | DHFR | tdk | thyX | Comments |
|---|---|---|---|---|---|
| Bacteria: | | | | | |
| Campylobacter | − | − | − | + | Food |
| Jejuni | | | | | poisonings |
| Helicobacter pylori | − | − | − | + | Formation of |
| | | | | | stomach ulcers |
| Rickettsia | − | − | − | + | Typhus causal |
| prowazekii | | | | | agent |
| Borrelia | − | − | − | + | Involved in |
| burgdorferi | | | | | Lyme's disease |
| Treponema pallidum | − | − | − | + | Syphilis causal |
| | | | | | agent |
| Chlamydia (3 | − | + | − | + | Compulsory |
| species) | | | | | intracellular |
| | | | | | pathogens |
| Mycobacterium | + | + | − | + | Tuberculosis |
| tuberculosis | | | | | |
| Archaebacteria: | | | | | |
| Pyrococcus abyssi | − | − | − | + | Hyperthermophilic |
| Pyrococcus | − | − | − | + | Hyperthermophilic |
| horikoshii | | | | | |
| Sufolobus | − | − | − | + | Hyperthermophilic |
| solfaraticus | | | | | |
| Eukaryotes: | | | | | |
| Dictyostellium | N.A | | N.A. | + | No complete |
| | | | | | genomic |
| | | | | | sequence |
| Virus: | | | | | |
| Bacterial and | − | | N.A | + | |
| eukaryotic DNA | | | | | |
| viruses (5 species) | | | | | |

Tdk: thymidine kinase required for recovering exogenous thymidine.
DHFR: dihydrofolate reductase.

It is to be noted that, in the Chlamydia sequence, the Serine residue is either absent, or is located at the aminoterminal end of the protein.

The differences in the enzyme mechanism of the two different classes of thymidylate-synthetases are also due to the absence of sequence patterns essential for the catalysis in the ThyA and ThyX proteins. Those data have made it possible to identify analogues of dUMP, dTMP, folate and flavin nucleotides as ideal candidates for key compounds for identifying novel compounds inhibiting the ThyX activity.

REFERENCES

ALSTCHUL S F, Madden T L, SCHAFFER M, ZHANG J, ZHANG Z, Miller W. & LIPMAN DJ Nucleic Acids Res. 25, 3389-3402 (1997).

Beaucage et al., Tetrahedron Lett 1981, 22: 1859-1862 Behr, Bioconjugate Chem., 5, 382-389 (1994)

Brown E L, Belagaje R, Ryan M J, Khorana H G, Methods Enzymol 1979;68:109-151

CARRERAS, C. W. and SANTI, D. V., 1995, Annu. Rev. Biochem. vol.64: 721-763.

DYNES J. L. and FIRTEL R. A., 1989, Proc. Natl. Acad. Sci. USA, vol.86: 7966-7970.

Fuller S. A. et al., 1996, Immunology in Current Protocols in Molecular Biology, Ausubel et al. Eds, John Wiley & Sons, Inc., USA

Green et al., Ann. Rev. Biochem. 55:569-597 (1986)

Houben Weyl, 1974, in Meuthode der Organischen Chemie, E. Wunsch Ed., Volume 15-I et 15-II,

Izant J G, Weintraub H, Cell 1984 Apr;36(4):1007-15

Koch Y., 1977, Biochem. Biophys. Res. Commun., 74:488-491

Kohler G. and Milstein C., 1975, Nature, 256: 495.

Kozbor et al., 1983, Hybridoma, 2(1):7-16.

Leger O J, et al., 1997, Hum Antibodies, 8(1): 3-16

Martineau P, Jones P, Winter G, 1998, J Mol Biol, 280(1):117-127

Merrifield R B, 1965a, Nature, 207(996): 522-523.

Merrifield R B, 1965b, Science, 150(693): 178-185.

Michaels, M. L. Kim, C. W. Matthews D. A. & Miller, J. H. Proc. Natl. Acad. Sci. USA 87, 3957-3961 (1990).

Myllykallio, H. et al., Science 288, 2212 (2000).

Narang S A, Hsiung H M, Brousseau R, Methods Enzymol 1979;68:90-98

PAPAMICKAEL D. 2000, STEM. Cells, vol.18.166-175.

Reimann K A, et al., 1997, AIDS Res Hum Retroviruses. 13(11): 933-943

Ridder R, Schmitz R, Legay F, Gram H, 1995, Biotechnology (N Y), 13(3):255-260

Roberts, D., Biochemistry 5, 3546 (1996).

Rossi et al., Pharmacol. Ther. 50:245-254, (1991)

Sambrook, J. Fritsch, E. F. and T. Maniatis. 1989. Molecular cloning: a laboratory manual. 2ed. Cold Spring Harbor Laboratory, Cold spring Harbor, New York.

Schofield, Brit. Microencapsulated. Bull., 51(1):56-71 (1995)

Sczakiel G. et al., 1995, Trends Microbiol., 1995, 3(6):213-217

Carreras C. W. and Santi D. V.,ANNUAL Review on Biochemistry, 1995, 64: 721-762.

Poole A., Penny D. and Sjöberg B. M._Nature Reviews, Mol. Cell. Biol. 2001, 2: 147-151

Papamichael D., 1999, The Oncologist, 4(6): 478-487.

TATUSOV R. L. GALPERIN, M. Y. Natale, D. A. & KOONIN, E. V. Nucleic Acids Res. 28, 33-36 (2000).

1. The use of a THYX polypeptide comprising the following amino acid sequence:
X₁HR(X)₇S, wherein:
X₁, represents the amino acid R (Arginine or Arg) or K (Lysine or Lys), and
(X)₇ is a chain with seven consecutive amino acids wherein each X represents, independently from each other, any of the 20 naturally occurring amino acids, in an in vitro synthesis method of the thymidine 5′-monophosphate (dTMP).

2. The use according to claim 1, characterized in that the THYX polypeptide is selected amongst polypeptides comprising amino acid sequences SEQ ID N^{o}1 to SEQ ID N^{o}37.

3. The use of a nucleic acid coding for a THYX polypeptide according to one of claim 1 or 2, for producing said THYX polypeptide.

4. The use according to claim 3, characterized in that the nucleic acid is selected amongst nucleic acids comprising nucleotidic sequences SEQ ID n^{o}44 to SEQ ID n^{o}64.

5. A method for screening a candidate molecule or substance interacting with a polypeptide according to the invention, said method comprising the steps of:
a) contacting a polypeptide according to one of claim 1 or 2 with the candidate substance or molecule to be tested;
b) detecting the complexes optionally formed between said polypeptide and said candidate substance or molecule.

6. A a set or a kit for screening a candidate molecule or substance interacting with a polypeptide such as defined in one of claim 1 or 2, said set comprising:
a) a THYX polypeptide such as defined in one of claim 1 or 2;
b) if need be, means necessary for detecting the complex being formed between said polypeptide and the candidate molecule or substance.

7. A method for screening an anti-bacterial or anti-viral compound in vitro in an acellular system, characterized in that said method comprises the steps of:
a) preparing a cell lysate from a culture of cells expressing a THYX polypeptide such as defined in one of claim 1 or 2 in the absence of a polypeptide coded by a thyA gene;
b) adding to the cell lysate obtained in step a) the inhibiting compound to be tested;
c) comparing the thymidylate synthase activity respectively in the cell lysate as obtained in step a) and in the cell lysate as obtained in step b); and
d) selecting the candidate compounds for which some inhibition of the thymidylate synthase activity has been detected.

8. A kit or a set for screening a thymidylate synthase inhibiting compound, characterized in that it comprises:
a) a composition comprising a THYX polypeptide such as defined in one of claim 1 or 2 in solution or under a lyophilized form;
b) optionally one or more reagents required for quantifying the thymidylate synthase activity.

9. The use of a nucleic acid coding a THYX polypeptide such as defined in one of claim 1 or 2 in a method for screening anti-bacterial or anti-viral compounds.

10. A kit or a set for screening an anti-bacterial or anti-viral compound, characterized in that it comprises:
a) a recombinant expression vector comprising a nucleic acid coding a THYX polypeptide such as defined in one of claim 1 or 2, under the control of a functional promotor in a host cell wherein its expression is being sought or in a host cell transfected with such a recombinant vector;
b) optionally one or more reagents required for quantifying the thymidylate synthase activity.

11. The use of an antisense oligonucleotide specifically hybridizing with a nucleic acid coding a THYX polypeptide such as defined according to one of claim 1 or 2 for inhibiting or blocking the transcription and/or the translation of said nucleic acid.

12. The use of an antibody directed against a THYX polypeptide such as defined according to one of claim 1 or 2 for inhibiting or blocking the thymidylate synthase activity of said polypeptide.

13. An anti-bacterial or anti-viral pharmaceutical composition comprising, as an active principle, an antisense oligonucleotide specifically hybridizing with a messenger RNA coding a THYX polypeptide such as defined in one of claim 1 or 2, in association with one or more physiologically compatible excipients.

14. The use of an antisense oligonucleotide specifically hybridizing with the messenger RNA coding a THYX polypeptide such as defined in one of claim 1 or 2 for producing an anti-bacterial or an anti-viral drug.

15. An anti-bacterial or anti-viral pharmaceutical composition comprising, as an active principle, an antibody specifically raised against a THYX polypeptide such as defined in one of claim 1 or 2 in association with one or more physiologically compatible excipients.

16. The use of an antibody specifically raised against a THYX polypeptide such as defined in one of claim 1 or 2 for producing an anti-bacterial or anti-viral drug.

17. The use of a nucleic probe or primer hybridizing with a nucleic acid coding a THYX polypeptide, such as defined in one of claim 1 or 2, in a method for detecting a bacterium or a virus.

18. The use of a nucleic acid coding a THYX polypeptide, such as defined in one of claim 1 or 2 as a selection marker for a genetic transfection, transformation or recombination event of a host cell or a host organism.

19. A reaction medium for the thymidylate synthase activity of the ThyX, characterized in that it comprises reduced flavins and CH₂H₄folate.

20. A medium according to claim 19, characterized in that the reduced flavins are obtained through reduction in situ of oxidized flavins.

21. A medium according to any of claim 19 or 20, characterized in that the oxidized flavin concentration varies from 50 μM to 1 mM, and is preferably 0.5 mM.

22. A medium according to any of claim 19 to 21, characterized in that the CH₂H₄folate concentration varies from 50 μM to 2 mM, and is preferably 1 mM.

23. A medium according to any of claim 19 to 22, characterized in that it additionally comprises dUMP in a concentration ranging from 1 μM to 800 μM, preferably 500 μM.

24. A medium according to any of claim 19 to 23, characterized in that the oxidized flavins are flavin mononucleotides and/or flavin adenine dinucleotides.

25. A medium according to any of claim 19 to 24, characterized in that the flavin reduction occurs through chemical, enzymatic, photochemical or electrochemical route.

26. A medium according to any of claim 19 to 25, characterized in that the flavin reduction occurs with NADH and/or NADPH.

27. A medium according to claim 26, characterized in that the NADH concentration ranges from 0.1 to 1 mM, and the NADPH concentration from 0.5 to 5 mM.

28. A method for screening an anti-bacterial or anti-viral compound in vitro in an acellular system, characterized in that said method comprises the steps of:
a) preparing a cell lysate from a culture of cells expressing a THYX polypeptide in the absence of a polypeptide coded by a thyA gene and comprising a medium according to one of claim 19 to 27;
b) adding to the cell lysate obtained in step a) the inhibiting compound to be tested;
c) comparing the ThyX thymidylate synthase activity respectively in the cell lysate as obtained in step a) and in the cell lysate as obtained in step b); and
d) selecting the candidate compounds for which some inhibition of the ThyX thymidylate synthase activity has been detected.

29. A screening method according to claim 28, characterized in that dTMP and ³H are used as markers for the ThyX thymidylate synthase ThyX activity.

30. A kit or a set for screening a ThyX thymidylate synthase inhibiting compound characterized in that it comprises:
a. a composition comprising a THYX polypeptide as well as a medium according to any of claim 19 to 27, in solution or under a freeze-dried form;
b. optionally one or more reagents required for quantifying the ThyX thymidylate synthase activity.

31. A kit or a set for screening an anti-bacterial or an anti-viral compound, characterized in that it comprises:
a. a recombinant expression vector comprising a nucleic acid coding a THYX polypeptide under the control of a functional promotor in a host cell wherein its expression is being sought or a host cell transfected with such a recombinant vector;
b. a medium according to any of claim 19 to 27;
c. optionally one or more reagents required for quantifying the ThyX thymidylate synthase activity.


SEQUENCE LISTING (64 sequences)

SEQ ID NO: 1 - protein, 265 aa, Borrelia burgdorferi
MNKEYKILDNGFLKLIDFMGDDRRIVKAARISYREESVKRKDAELIDYLIRNGHTSPLEQVVFTFHVKAPIFVARQWMRHRTARINEVSGCYSLAREEFYVPLEEDLKCQTSSNSSEKEFKSLEKLSDKIKHHQKHSYELYQDMINANIPKELSRIVLPLSLYTEWYWQIDLNNLFHFIKLRLALDSPKEIKENSPKEMREYAKALISIVREIVPIAFNSFENHFLRGKRFSHEEIIAIINALDLNKLSMDAEKLNLLKDKLGID

SEQ ID NO: 2 - protein, 305 aa, Treponema pallidum
MTLRTLQAGVAVSIALDRVCFFCYNGAVAHCVVEAAEDILDRRFSVLDKGFVRLIDYLGGDARIVQAARVSYGAGTRTARDDAALIDFLLRNKHTSPFEQVVLTFHVRAPIFVARQWMRHRTARISEVSSRYSLLSHDCYVPQETSVAVQSTRNKQGRASEGISPEQQQEVRAAFEAQQKAACAAYDALIQKNIARELARINVPLSLYTEWYWQIDLHNLFHFLRLRASAHAQAEIRAYAEVIIEITRAVAPCATASFENHEKDGVQFSGREFAALKALLAGEGLSLEGKERARFEEKLRSGLQQ

SEQ ID NO: 3 - protein, 294 aa, Rickettsia prowazekii
MHNTTKRVTVPALEAMLYETIKVLDHGFIRVIDYMGDDSSIVQAARVSYGKGTKQLNQDKGLINYLLRHYHTTPFEMCDIKFHIKLPIFIARQWIRHRTASVNEYSARYSILGNEFYLPDPANIASQSVVNKQCRAGDSVPKKVSEKVLAILEEDARRCYRHYKELMNADEDGNILDENVSGIARELARINLTLNYYTEWYWKINLHNLLHFLRLRTDPKAQYEIRVYAEKILDIVKAWVPFTYEAFEEYRLQGANISRKGLEVIKRMIKGEKVIHETSGMNKREWEELVKIFR

SEQ ID NO: 4 - protein, 220 aa, Thermogata maritima
MKIDILDKGFVELVDVMGNDLSAVRAARVSFDMGLKDEERDRHLIEYLMKHGHETPFEHIVFTFHVKAPIFVARQWFRHRIASYNELSGRYSKLSYEFYIPSPERLEGYKTTIPPERVTEKISEIVDKAYRTYLELIESGVPREVARIVLPLNLYTRFFWTVNARSLMNFLNLRADSHAQWEIQQYALAIARIFKEKCPWTFEAFLKYAYKGDILKEVQV

SEQ ID NO: 5 - protein, 260 aa, Dictyostelium discoideum
MGLDIQTEIDKIVIEKVKPEVEYYDVMGGSHRWEVKVHDHGKVALVDTMPRLAPVGQTADFSICQAARVSYGAGTKKVTEDKGLIRYLYRHQHTSPFEMVEFKFHCVMPVFIARQWIRHRTANVNEYSARYSVLPDKFYHPSIEEVRKQSTSNRQGGEEALEPKTAQEFLDYLDKVEENYKTYNELLEKGLSRELGRIGLPVSIYTEWYWKIDLHNLFHFLRLRMDSHSQKEIRDYANTIFALIRPIVPVACEGIYRLCF

SEQ ID NO: 6 - protein, 222 aa, Roseophage
MTQIEATYIDHMGSDLSVVNAARVSFGKKSEWVYCGQSDGRDKGLSGRDTKLIKYLAKHKHISPFGHAFASFHVKAPIFVARQLVKHKFLRWNEISRRYVDDEPEFYTPDVWRGRSADKKQGSDGVVNPEYNPQYLDNKIKFAYLQALDIGISPEQARMLLPQSTMTEWYWSGSLDAFADMCRLRCKEDTQYESRVVADQISEKMADLYPVSWAALMEGEKQ

SEQ ID NO: 7 - protein, 246 aa, Streptomyces coelicolor
MTDIPADDPKIELRSDITVELVKSAATDSDVLFAARVSTAGEQSLDELKKDPERSKGLINYLMRDRHGSPFEHNSMTFFVSAPIFVFREFMRHRVGWSYNEESGRYRELQPVFYAPDASRKLVQQGRPGKYVFVEGTPEQHELVGSAMEDSYRQAYATYQQMLAAGVAREVARAVLPVGLYSSMYATCNARSLMHFLGLRTQHELAKVPSFPQREIEMAGEKMEAEWARLMPLTHAAFNANGRVAP

SEQ ID NO: 8 - protein, 317 aa, Aquifex aeolicus
MMKIYLMGSDQRIVRCARVSFAKDSYVDEKRDKRLIRYLFKHRHASPFEHNIIAFEWKKEKWIELLSKLENPTVQVYYSNGFVFLNLRNAINVWELLPDAVKERIKEAFPTTYGVIQRRGEIEDEELYSLPYTKDKAYVKEKIETSSGWIGLVDKLELETDMDFYTFVVECPLFVARQWMRHRFGSYNEVSKRYVGKEFLEFYLPKYIRKQAEKNKQASVDEPISESEVFIKKIENLISKSVKLYEEIIEKGGAKELARGVLPQFMKTRFYWTVPRISLDNFITLRTHEGAQKEIREFAEAIKEMVGYRGTDKKNVI

SEQ ID NO: 9 - protein, 216 aa, Chorella virus
MSAKLISVTKPVVEGVNTAEELIAYAARVSNPENQINNKTASGLLKYCIRHKHWSIFETAFMTLELKTSRGIAAQVLRHRSFHFQEFSQRYASVMETPPPHQARFQDHKNRQNSLDTVPEDDQTWWATEQEKLYAQSMELYNKALEKGIAKECARFILPLSTPTTIYMSGTIRDWIHYIELRTSNGTQREHIDLANACKEIFIKEFPSIAKALDWV

SEQ ID NO: 10 - protein, 215 aa, Synechocystis sp.
MDVRFISLTKPEIVIDGEPLSPEGLIAYCARVSSPNQENPNYTKLLQFCIREGHWSIFEMVDMTLEITTTRAIAPQILRHRSFSFQEFSLRYSCATEYECYEARRQDVKNRQNSLDDFDESTKKWFNQAQAAVWEKSHQLYEEALAKGIAKECARSILPLNTVTRLYMKGSVRSWIHYFSVRCDQATQKEHREIALAARKIFMKHFPTVAAALEW

SEQ ID NO: 11 - protein, 243 aa, Pyrococcus horikoshii
MVKVKLINYTPKPLETVTWAALISYWDGWSTEAFEKISPNDVEIHLPRILSYGHESILEHATFTFSIEGCSRVCTHQLVRHRIASYTQQSQRYIKINPEDVEETFVIPESIKKDSELLKEWKELLKRSLELYEKSIERGIHQEDARFILPQSVKTKIVVTMNLRELKHFFGLRLCERAQWEIREVAWKMLEEIAKRKELKPIIEWAKLGPRCIQLGYCPERELMPPGCLKRTRERWKNLLEKY

SEQ ID NO: 12 - protein, 244 aa, Pyrococcus abyssi
MVRVTLVNYTRRPLETITWAALVSYWDEWSTESFEKINEDDVKAHLPRILGYGHESILEHATFTFSIEGCSRVCTHQLVRHRIASYTQQSQRYIVLNEENVEETFVIPESIKKDRELYEKWKKAMAETIKLYKESLKRGIHQEDARFILPQAVRSKIVVTMNLRELKHFFGLRLCERAQWEIREVAWKMLEEIAKREELRPIIKWAKLGPRCIQLGYCPERELMPPGCFKRTRERWMKLLEKPL

SEQ ID NO: 13 - protein, 266 aa, Halobacterium sp.
MVPARGFGVFLPPAGTPSSMRVRLLEATENPEELICQSARNDYMSDWVGDTPLDTAMASVDGDTTDEKLSNLIAQLLTRGHYGPFEHPSATFAIEGVSRSCMAQLTRHRHASFDVQSMRYVAFDDVDPAAVAEGELVVTPPSATDPDWVGRNQDAGDIDEETMAEREAVFQASVRRAVEDYQELLGLGMPPEDARFVLPIGTEVNVVITLNPRSLMHVADMRAAADAQWEIRELTEQLLDAAAQWCPHTFEYYDAEMKHRKNRLAP

SEQ ID NO: 14 - protein, 235 aa, Mycobacteriophage D29
MKVQLIASTILEDPSWAGTDYVGDDETVTSADELAEFAGRNCYLSFDRPNPKTRENVDYLNHILDVGHESVLEHSSATFYIEASRSVLTELERHRHLSFSVVSQRYVDPTELGIHVPPAFTELSGSDADKAKEVLLDVQSFAQEAYEYLVHIFSDAGFPRKKAREAARAVLPNMTNSPMVVTGNHRAWRYVIKNRWHEAADAEIRELAGELLRQLREIAPNTYQDIPTEPYSYGG

SEQ ID NO: 15 - protein, 250 aa, Mycobacterium tuberculosis
MAEQVKLSVELIACSSFTPPADVEWSTDVEGAEALVEFAGRACYETFDKPNPRTASNAAYLRHIMEVGHTALLEHANATMYIRGISRSATHELVRHRHFSFSQLSQRFVHSGESEVVVPTLIDEDPQLRELFMHAMDESRFAFNELLNALEEKLGDEPNALLRKKQARQAARAVLPNATESRIVVSGNFRTWRHFIGMRASEHADVEIREVAVGCLRKLQVAAPTVFGDFEIETLADGSQMATSPYVMDF

SEQ ID NO: 16 - protein, 250 aa, Corynebacterium glutamicum; identical to SEQ ID NO: 15

SEQ ID NO: 17 - protein, 243 aa, Mycobacteriophage l5
MKAKLIAATEIDPGALRDIGFEVDDFEESKDEDPYFGDFDADELAEFAGRNCYRSFHRPNPATAENEDYLNHIIDLGHESVFEHASATFYIEASRSVLTELERHRHLSFSVVSQRYVDPTDLGIHLPPALFKLHPDDRDDLVHIMESVSSEIDAVYEHIVNRLADRGLPRKQAREAARAVLPNMTNSPMVVTGNHRAWRYVIKARWHEAADAEIRELAGELLRQLRQIAPNTYQDIPDVPYSY

SEQ ID NO: 18 - protein, 237 aa, Bacteriophage phi-C31
MKVNVLATTALNPSPLLDAYEYRVSGAAYNRDRPTDADALGEAAGRICYKSFERKNPATASNPGYLGNILAQGHFSVLEHASVTFLVRDVSRALLTELSRHRHLSFSVVSQRYVDHADTEPVVPPAIRGTELEKPFREDYAEALQAYDAGVKLLRARGYGRKQAREAARALLPNAAPVDMVVTGNLRAWRDVLGKRWHVAADAEIREFAGRVLDHLHAVAPNSVQDMPTSPFGSDGK

SEQ ID NO: 19 - protein, 327 aa, Aeropyrum pernix
MSLAASLEKAGLGISVRLLEYTGDGERIVAVASKVSLSRSPAERLLAIGEDEVETWILETFRRQHFSPWEHSVYTFMVEGLSRVASHQLVRHRVASYTQLSHRYSEGYLREAALKACESIGLDCPSKPAETEGGRKAAYRLYSQALERAARDFGASERFAIAAKAFVIPPTILARGDGGDGVVEAYLRSAAIYYSLLSRGARREDARYILPDALRTRIVVTMNARELIQVFFPLRMCTRAQWEIRHIAWLLWRELSRVHPRLFRWAGPSCVLRENTLRTTPASLYSYLEGVERFTQPRCPELVENKAIPGCLRQAASVAPPGDGEYE

SEQ ID NO: 20 - protein, 269 aa, Treponema denticola
HSPMAHCIAPEAEKILDKEFKVLDKGFIRLVDYMGTDARIVQSARVSYGEGTKTVREDAALIDYLLRNKHTSPFEQVVFTFHVKLPIFVARQWIRHRTARLNEISGRYSILKAEFYVPAGKDIALQSSDNKQGRMNEAVPQDLQNEVITSLQKQQEEIYAGYSKLLDKNIARELARINLPLSTYTEWYWQIDLHNLFHFLRLRMDAHAQKEIRDYAEVMFEICKTVTPLACASFERHEKNGVNFSAEELEAIRNLIAGKDSGLKGKELE

SEQ ID NO: 21 - protein, 231 aa, Helicobacter pylori
MWITQETWLKALPWNKKRYRSQIMEVICKHYTPLDIASQAIRTCWQSFEYSDDGGCKDKELIHRVGNIFRHSSTLEHLYYNFEIKGLSRGALQELSRHRIASLSVKSSRYTLRELKEVESFLPLNETNLERAKEFLVFVDNEKVNAMSVLALENLRILLSEHNIKNDLAKYAMPESYKTHLAYSINARSLQNFLTLRSSNKALKEMQDLAKALFDALPGEHQYLFEDCLKH

SEQ ID NO: 22 - protein, 241 aa, Pyrococcus furiosus
MVRVTLVNYTKRPLETITWAALISYWGEWSTESFERISENDVEKHLPRILGYGHESILEHATFTFSIEGCSRVCTHQLVRHRIASYTQQSQRYIVLDEENVEETFVIPESIKKDRELYEKWKKVMAETISLYKESINRGVHQEDARFILPQAVKTKIIVTMNLRELKHFFGLRLCERAQWEIREVAWKMLEEMAKRDDIRPIIKWAKLGPRCIQFGYCPERDLMPPGCLKKTRKKWEKVAE

SEQ ID NO: 23 - protein, 282 aa, Rhodobacter capsulatus
TTLRATSAGMEARLYQAHPVLDHGLIRVIDYMGDDAAICQAARVSYGRGTKAVSDDRGLIRYLMRHWHSTPFEMCEVKFHVKLPIFVARQWIRHRTANVNEYSARYSVMDREFYIPAPEHLAAQSTVNNQGRGQVLEGAEAARVLDLLREDAMRAYDHYEDMLTPDADAGKLGLARELARMNLPANVYTQWYWKIDLHNLFHFLRLRADVHAQYEIRVYAQIMGDIVKDWVPQAYEAFEDYRLGAVSVSAKAKEVLKRRLAGEVVTAETSGMSKGEWREFVE

SEQ ID NO: 24 - protein, 247 aa, Halobacterium salinarum
MRVRLLEATENPEELICQSARNDYMSDWVGDTPLDTAMASVDGDTTDEKLSNLIAQLLTRGHYGPFEHPSATFAIEGVSRSCMAQLTRHRHASFDVQSMRYVAFDDVDPAAVAEGELVVTPPSATDPDWVGRNQDAGDIDEETMAERQAVFQASVRRAVEDYQELLGLGMPPVDARFVLPIGTEVNVVITLNPRSLMHVADMRAAADAQWEIRELTEQLLDAAAQWCPHTFEYYDAEMKHRKNRLAP

SEQ ID NO: 25 - protein, 254 aa, Mycobacterium leprae
MAQIAPLRVQLIAKTEFLAPPDVSWTTDADGGSALVEFAGRACYQSWSKPNPRTATNAAYIKHIIDVGHVAVLEHASVSFYISGISRSCTHELIRHRHFSYSQLSQRYVPEKDAQVVVPPDMEDDDELQQILIAAVEASRATYTELLVKLNAKLMAGELGGNRAVLRRKQARQAAHAVLPNANETRIVVTGNYRAWRHFIAMRASEHADVEIRRLAIVCLRRLVDVAPAVFADFEITALADGTEVATSPLATEA

SEQ ID NO: 26 - protein, 208 aa, Helicobacter pylori
MEVICKHYTPLDIASQAIRTCWQSFEYSDDGGCKDRDLIHRVGNIFRHSSTLEHLYYNFEIKGLSRGALQELSRHRIASLSVKSSRYTLRELKEVESFLPLNETNLERAKEFLVFVDDEKVNEMSVLALENLRVLLSEHNIKNDLAKYAMPESYKTHLAYSINARSLQNLLTLRSSNKALKEMQDLAKALFDALPYEHQYLFEDCLKH

SEQ ID NO: 27 - protein, 207 aa, Campylobacter jejuni
MQITLLFHTPLSVCSHATRTCWQSFEKGDCGGEKDKELIDRVGNKFKHASTLEHLNYTFYIQGISRACLQEVARHRHTSPSVKSTRYTLKELRNEAEFKIGDFENASRYLVLCGNEEVDNASIKALENLRTILQKSISLDIAKYCLPESYKTELTLTINARSLQNFISLRSSKSALWEIRNLANALFEALPQEHKFIFEHCLHKDIE

SEQ ID NO: 28 - protein, 260 aa, Sulfolobus solfataricus
MISVKLVSYTNDGEKVIAIAAKMSRSRKGWDYHEKDMTDDEIETWIRDAILHGYWSVLEHSVYTFSIEEISRVASHQLVRHRIASYTQMSHRFAKPIDEYYKPIIPPSAKKRSKELVEKAYKEAYDNYYTLLESGVPEEDARYVLPNGVNTNIVVTMNARELYNFFSLRLCSRAQWEIRAIAWKMLEEVKKVHPRLFKYTGPNCIIHENFIRNENESITLEDIFKDYKLEFISQRCIEGVLRDGIKKCIINSRSVLDNIK

SEQ ID NO: 29 - protein, 250 aa, Mycobacterium bovis
VAETAPLRVQLIAKTDFLAPPDVPWTTDADGGPALVEFAGRACYQSWSKPNPKTATNAGYLRHIIDVGHFSVLEHASVSFYITGISRSCTHELIRHRHFSYSQLSQRYVPEKDSRVVVPPGMEDDADLRHILTEAADAARATYSELLAKLEAKFADQPNAILRRKQARQAARAVLPNATETRIVVTGNYRAWRHFIAMRASEHADVEIRRLAIECLRQLAAVAPAVFADFEVTTLADGTEVATSPLATEA

SEQ ID NO: 30 - protein, 251 aa, Corynebacterium diphtheriae
MQQSLLDVQLVACSTFTTPSGVDWKVDSAATDSEALVEFAGRACYETFDKPNPRTAANDAYIRHIMEVGHMALLEHPTATVYIRGLSRSATHELVRHRHFSFSQLSQRFVHSDETHVVIPPLIDNDPQLRELFLSTVDEVRFAYSELMTALDNKLADEPNAILRRKQARQAARSILPNATESRIVVTGNFRAWRHFIGMRATEHADVEIRSLAVRCLEILKEKAPTVFSDFETSVLSDGSIMATSPYVTDY

SEQ ID NO: 31 - protein, 161 aa, Bacillus anthracis
DRLIRHIVGSGHASTLEHLTYTFAVEGVSRALLAQLTRHRVGFSYSVQSQRYVRMGSNDKIGGFDYVVPETVKAKGEQVVNAYNEMMYKLQSGYDLLRTLGIPAEDARSVLPNAAATNLVLTVNLRGLLDFYNKRRKGKGAQAEIAELAEQLRQEVVKVEK

SEQ ID NO: 32 - protein, 212 aa, Clostridium difficile
KLISHTPEPEKVIAMAAKLCYSPVGTDEIEKDLTDESIEKFLNMLLSIGHGSILEHASFTFSIEGISRACSHQIVRHRIASFSQQSQRYVKLEQFEYIIPPEIEKELFIDSMKKDQENYDKLVEILFENHYNDLIKNGKNEKTAKRQAEKKAIEDARYVFPNACETKMVFTINARSLFNFFEHRCCERAQWEIRNLAVEMLREVKKVAPILF

SEQ ID NO: 33 - protein, 515 aa, Thermoplasma volcanium
MEFSNAERDVFLIKIEKMIDRGALMSRYSRASDPDIRSVYEKEFKTGAKSGEEFYRRIFLEYGDESIAELTTAQMGIQNVSNVASKIIEEIRIGLSYLEKSTRYVRYDKKVDGRYLYISPERIGISGDDAKDYVQLCDNLFEFYSKALPQVEDYLRHKFPQDKLVFQNAGGKTLTEMDQNEKKIAERSYINAVRSRALDDVRYILPASTLTNIGISGNGRALIHLIQKLMEYEIPETTKLAKDIYDELKPELPQLIDDALSGHGLEIINFKKNLMGLFPYDLTGNFERIRLLSYGEEDKELRKVASLIEYPFHGDAASLYSRSSESYVKYMKELIESIRSLRANRRMKPGRAFESVNYVFELNLNYGSFRDLQRHRFLGIIRKPLTAAYGYDTPPVISAIDELKTQYDELMANSSSFYQRLREKYGPWISQYVVPFAFKYPITFSTNLSEVTYFVELRSTAQAHFDLRDIAVSMYREVSKVHPTLSRIIKFVDTADYPLGRLSAEFRKESKKAGI

SEQ ID NO: 34 - protein, 499 aa, Thermoplasma acidophilum
MIDRGALMSRYSRASDPDIRSVFHREFEGNQKRSEDFYRRIFLEYGDESIAELVTAQVGIQNVSNVISKVIEEIRIGLSYLEKSTRYVAYDRKVDGHYLFMQAEKIGLSGEAAREYTDLCNRLFDLYSSTLPRIEEEISRQWPIESFDFNIDGNPRNYKELDENGRKLAQKSYRSSVRSRALDDARFILPASTLTNMGVSGNGRSFIHLIQKLMEYGVPESERLAHDLYEELKGEFPQIIDDALSQHGQDIINYKRSLASLFPYTDGGRFEKVRLIKYSNEREEMQKVLALLMYPFAEDASGIISRIKAMELSEASAILERIRDLRKNRRMKVGRPFEAVNYVFEVTTNYGAFRDLQRHRFLSIVRKPLTVSYGFDVPPIIAKMPDLSEEYAEAMKDAERVYRIIKERYGAWIAQYAVPFAYRYPVVFTTNLAEATYFIELRSTPQAHFDLRDIAIRMYNEIKSVHPSLAGIIKFVDTGDYPLGRLSAEVRKNVKAGGI

SEQ ID NO: 35 - protein, 531 aa, Chlamydophila pneumoniae
MLGKEEEFTCKQKQCLSHFVTNLTSDVFALKNLPEVVKGALFSKYSRSVLGLRALLLKEFLSNEEDGDVCDEAYDFETDVQKAADFYQRVLDNFGDDSVGELGGAHLAMENVSILAAKVLEDARIGGSPLEKSTRYVYFDQKVRGEYLYYRDPILMTSAFKDMFLGTCDFLFDTYSALIPQVRAYFEKLYPKDSKTPASAYATSLRAKVLDCIRGLLPAATLTNLGFFGNGRFWQNLIHKLQGHNLAELRRLGDESLTELMKVIPSFVSRAEPHHHHHQAMMQYRRALKEQLKGLAEQATFSEEMSSSPSVQLVYGDPDGIYKVAAGFLFPYSNRSLTDLIDYCKKMPHEDLVQILESSVSARENRRHKSPRGLECVEFGFDILADFGAYRDLQRHRTLTQERQLLSTHHGYNFPVELLDTPMEKSYREAMERANETYNEIVQEFPEEAQYMVPMAYNIRWFFHVNARALQWICELRSQPQGHQNYRTIATGLVREVVKFNPMYELFFKFVDYSDIDLGRLNQEMRKEPTT

SEQ ID NO: 36 - protein, 529 aa, Chlamydia trachomatis
MLSKEGGFSEEQRARLSHFVTNLDSPIFALKNLPEVVKGALFSKYSRSTLGLRALLLKEFLDGEGGNFLDDDQQDCELGIQKAADFYRRVLDNFGDDSVGELGGAHLALEQVSMLAAKILEDARIGGSPLEKSSRYVYFDQKVNGEYLYYRDPILMTSAFKDVFLDTCDFLFNTYSDLIPQVRSHFEKLYPKDPEVSQSAYTVSLRAKVLDCLRGLLPAATLTNLGFFGNGRFWQNLLHRLQDNSLVEVRNIGEQSLTELMKIIPSFVSRAESHHYHHQAMVDYRRALKEQLKSFAHRYGEEREISKEAGVKLVYGDPDGLYKIAAAYMFPYSEHTYAELLDICRNIPNEDLMRILESGASFRENRRHKSPRGLECAEFAFDITADFGAYRDLQRHRILTQERQLLTTKLGYTMPSQLIDTPMEAPFREAMEKADQAYRLIAEEFPEEAQYVVPLAYNIRWLFHINARGLQWLCELRSQPQGHESYRKIAIDMAREVIQFHPAYELFLKFVDYSETDLGRLQQESRKKS

SEQ ID NO: 37 - protein, 529 aa, Chlamydia muridarum
MLSKEGDFSKEQRERLSHFVTNLDSPIFALKNLPEVVKGALFSKYSRSILGLRALLLKEFLDGEGGDFLSEDLQDCELGIQKAADFYRRVLDNFGDDSVGELGGAHLAIEQVSMLAAKVLEDARIGGSPLEKSSRYVYFDQKVNGEYLYYRDPILMTSAFKDTFLDTCDFLFNTYSELIPQVRAYFEKIYPKDPEVSQSAYTVSLRAKVLDCLRGLLPAATLTNLGFFGNGRFWQNLLHRLQDNNLVEVRNIGEQALTELMKIIPSFVSRAEPHHHHHQAMVDYHLGLREQLKSFAQRYGEEREPSLEKGVKLVYGDPDGLYKIAAASMFPYSEHTYADLLDICRKIPDEDLMLILESSASSRENRRHKSPRGLECAEFAFDITADFGAYRDLQRHRILTQERQLLTTKLGYSIPQQLLDTPMEAPFREAMEKADQAYRLIAAEFPEEAQYVVPLAYNIRWLFHINTRGLQWLCELRSQPQGHESYRQIAIDMAKEVIQFHPAYKSFLKFVDYSETDLGRLKQESRRKA

SEQ ID NO: 38 - DNA, 24 bp, Artificial sequence
  Description of artificial sequence primer
gccatggggatcacccaagaaact

SEQ ID NO: 39 - DNA, 24 bp, Artificial sequence
  Description of artificial sequence primer
atgcttcaaacaatcttcaaacaa

SEQ ID NO: 40 - DNA, 24 bp, Artificial sequence
  Description of artificial sequence primer
gccatggttcgagttacgctcgtc

SEQ ID NO: 41 - DNA, 24 bp, Artificial sequence
  Description of artificial sequence primer
taagggtttttcgagaagtttcat

SEQ ID NO: 42 - DNA, 24 bp, Artificial sequence
  Description of artificial sequence primer
gccatggaaatcactttacttttt

SEQ ID NO: 43 - DNA, 24 bp, Artificial sequence
  Description of artificial sequence primer
ttctatatctttatgtaggcaatg

SEQ ID NO: 44 - DNA, 693 bp, Helicobacter pylori
atgtggatcacccaagaaacttggctaaaagcgttaccgtggaataaaaagcgatataggagtcaaataatggaagtgatttgtaagcactatacccctttagacattgcgagccaagcgatccgcacttgctggcagagctttgaatacagcgatgatggaggctgtaaggataaagaattgatccacagggtggggaatatttttaggcattcttccactttagagcatctttattacaattttgaaatcaagggtttgagcaggggggcgttgcaagaattgagccggcatagaatagcgagcttgagcgtgaaatcaagccgttacactttgagggaattgaaagaagtggagagctttttaccccttaatgaaacgaatttagaaagagcgaaagagtttttagtttttgtggataatgaaaaagtgaatgcaatgagcgttttagctttggaaaatctaaggatcttattgagcgagcataacattaaaaacgatttagccaaatacgccatgcctgaaagctataaaacgcatttggcctatagtattaacgctaggagcttgcaaaatttcttgactttaagaagcagtaataaagccttaaaagaaatgcaagatttagccaaagccttatttgacgctttacctggcgagcatcagtatttgtttgaagattgtttgaagcat

SEQ ID NO: 45 - DNA, 624 bp, Helicobacter pylori
atggaagtgatttgtaagcattacacccctttagacattgcgagccaagcgatccgcacttgctggcagagttttgaatacagcgatgatggaggttgtaaggatagggatttgatccacagggtggggaatattttcaggcattcttccactttagagcatctttattacaattttgaaatcaagggtttgagtaggggggcgttgcaagaattgagccgacatcgaatagcgagcttgagcgtgaaatcaagccgttacactttaagggaattgaaagaagtggagagctttttaccccttaatgaaacgaatttagaaagagctaaagagtttttagtttttgtagatgatgaaaaagtgaatgaaatgagcgttttagctttggaaaatctcagggttttattgagcgagcataacattaaaaacgatttagccaaatacgccatgcctgaaagctataaaacgcatttagcctatagcattaacgctaggagcttgcaaaatttattgactttaagaagcagcaataaagccttaaaagaaatgcaagatttagccaaagccttatttgacgccttaccttatgagcatcaatatttgtttgaggattgtttgaagcac

SEQ ID NO: 46 - DNA, 621 bp, Campylobacter jejuni
atgcaaatcactttactttttcacactccattatctgtttgttctcatgcaacaagaacttgttggcaaagctttgaaaaaggcgattgcggtggcgaaaaagataaagaacttatcgatcgcgtgggtaataaattcaaacacgcttcaaccttagaacatctaaactatactttttatatacaaggaatttcaagagcttgcttacaagaagttgcaagacaccgtcacactagtcctagtgtaaaaagcacgcgttataccttaaaagaacttcgcaatgaagctgaatttaaaataggagattttgaaaatgcaagccgttatcttgtgctttgtggtaatgaagaggttgataacgcaagcattaaagccttagaaaatttacgcactattttacaaaaaagcattagtctagatatagccaaatactgcttaccagaaagctataaaaccgaacttacactaacgattaatgcaagaagtttacaaaattttatatctttgcgtagttcaaaatcagctctttgggagattagaaatttagcaaatgctttatttgaagccttaccacaagaacacaaatttatatttgaacattgcctacataaagatatagaa

SEQ ID NO: 47 - DNA, 882 bp, Rickettsia prowazekii
atgcacaatacgacaaaaagagtaacagtaccagcacttgaagcaatgttatatgagactatcaaagttttagatcatggttttattagggtaatcgattatatgggtgatgatagttcgatagtacaggcagctcgtgtttcttatggtaaaggtactaagcagttaaaccaagataaaggattaataaattacttgctgcgtcattatcatacgacaccttttgagatgtgtgacatcaaatttcacattaaattacctatatttattgcacggcagtggattaggcataggactgctagtgttaacgaatattcagcaaggtattctattttaggaaatgaattttatttacctgaccctgccaatattgcttctcaatctgttgtaaataaacaatgtagagcaggggatagcgtaccgaaaaaagtatctgaaaaagttctcgcaattttagaagaagatgctagacggtgctacaggcattataaggagctcatgaatgctgatgaagatggaaatattctagatgagaatgtttcaggcatagcaagagaacttgctcgtattaatttaactttgaattattatacggaatggtattggaagattaatttacataatttgcttcattttttaagattacgaactgatcctaaggcacaatatgaaattagagtttatgccgaaaagatacttgacatagtaaaagcttgggtaccttttacttacgaagcgtttgaagagtatcgtttgcaaggcgcgaatatttcacgtaaaggtttagaggtaattaaaagaatgataaaaggtgaaaaagtgatccatgaaactagtggtatgaataaaagagaatgggaggaattggtaaagatttttagg

SEQ ID NO: 48 - DNA, 660 bp, Thermotoga maritima
atgaagatcgatatcttggacaaaggattcgttgaacttgtggatgtgatgggaaacgacctctccgctgtacgggctgcccgcgtttctttcgatatgggtttgaaagacgaagaaagggatagacatctcatcgaatatctcatgaaacacggtcacgagacacctttcgaacatattgtcttcactttccacgtgaaagcccctatattcgtggcgaggcagtggttcagacacaggatcgcttcttacaacgaactgagcggcagatactcgaagctctcctacgaattctacattccctctcccgaacgcctggaagggtacaaaacgaccatccctcccgaacgggtgacggagaagatctcagaaatagtcgataaagcgtatcgaacgtatctggagttgatagaaagcggtgttcctcgagaagtggcaaggatagtgctccctttgaatctgtacacgaggtttttctggactgtgaacgcaagaagtctcatgaactttttgaacctgagggcagattctcacgctcagtgggaaattcaacagtacgctctggcaatcgcgagaattttcaaagagaagtgtccctggacttttgaggcatttcttaagtatgcttataaaggagatatcctgaaggaggtacaggta

SEQ ID NO: 49 - DNA, 729 bp, Pyrococcus horikoshii
atggttaaggtaaaactgataaattacactcccaaacccttggagacagttacttgggctgcacttataagctattgggatggttggagtaccgaagcttttgagaagatatcccctaatgatgttgaaattcacctacctagaatcctaagctacggacacgaatcaatcctggagcatgcaactttcacattctccatagagggatgctcaagggtgtgcacgcatcagctcgttaggcataggatagcaagttacacccaacagagtcaaaggtacattaaaataaaccctgaagatgttgaggaaaccttcgtcataccggagtccataaaaaaagattcggagttattaaaagagtggaaagagctacttaagagatctttagaattgtacgagaaaagcattgaaagggggatacatcaagaggatgcaagattcatccttccccaatcagttaaaacgaaaattgtagtaacgatgaacctaagggaactaaagcacttctttggattgagattatgcgagagggcccaatgggagatcagggaagttgcttggaagatgcttgaggaaattgccaaaaggaaagagctcaagccgataattgagtgggctaagctagggccaagatgcatccaacttggctactgtccagagagggaacttatgccaccaggttgcttaaaaagaacgagggaaaggtggaaaaacttattggaaaagtat

SEQ ID NO: 50 - DNA, 750 bp, Mycobacterium tuberculosis
gtggccgagaccgcgccgctgcgcgtgcaactgatcgccaagaccgacttcttggccccacccgacgtgccctggaccaccgacgccgacggcggacccgcgctggtcgagttcgccggccgggcctgctatcagagctggtccaagcccaatcccaagaccgccaccaacgccggctacctccggcacatcatcgacgtcggacatttctcggtgctagagcatgccagcgtgtcgttctacatcaccgggatctcgcgatcgtgcacccacgagctgatccgccaccggcatttctcctactcgcagctctcccagcgctacgtacccgagaaggactcgcgggtcgtcgtgccgcccggcatggaggacgacgccgacctgcgccacatcctgaccgaggccgccgacgccgcccgcgccacctacagcgagctgctggccaagctggaagccaagttcgccgaccaacccaacgcgatcctgcgccgcaagcaggcccgccaagccgcccgcgcggtgctgcccaacgccaccgaaacccgcatcgtggtgaccggcaactaccgggcctggcggcacttcatcgcaatgcgggccagcgagcacgccgacgtggaaatccggcgactggccatcgaatgcctgcgccagctcgccgccgtggcccccgcggtgttcgccgacttcgaggtgaccaccctggccgacggcaccgaggtggcgaccagcccgttggcgaccgaagcc

SEQ ID NO: 51 - DNA, 750 bp, Mycobacterium tuberculosis; identical to SEQ ID NO: 50

SEQ ID NO: 52 - DNA, 762 bp, Mycobacterium leprae
gtggcccagatcgcgccgctacgcgtgcaactaattgccaagactgagtttctggcgcctcctgacgtgtcgtggactaccgacgccgacggtggttccgcgctggtcgaattcgccggtcgcgcctgttatcagagctggtcgaagcccaatccccggaccgcgaccaacgccgcgtacattaaacacatcattgacgtcgggcatgttgcggtgctcgagcatgccagcgtttcgttctatatcagcggcatctcgcgatcatgcactcacgagctgatccgacatcggcatttctcctactctcagctgtcgcagcgctacgtgccggaaaaggatgcccaggtcgttgtgccaccggacatggaggatgacgacgaacttcaacagattctgattgcggccgtagaggccagccgggccacctacactgaactgctggtcaagctgaacgccaagttaatggccggtgagctcggcgggaatagggcggtgttgcggcgcaagcaagctcgccaagctgcccacgcggtgctgcccaacgccaacgagacccggatcgttgtgaccgggaactaccgggcatggcggcacttcatcgccatgcgggccagcgagcacgccgacgtcgaaatccggcggctggccattgtctgcctgcgccggctcgtcgacgttgcgcccgcagtattcgctgatttcgagatcaccgcacttgctgatggtactgaggtcgcaactagtcctttagccaccgaagct

SEQ ID NO: 53 - DNA, 780 bp, Sulfolobus solfataricus
atgatctctgttaaattagtctcatatacgaatgatggagagaaagtcattgctattgctgctaagatgagtaggagtagaaaaggttgggattatcatgagaaagacatgactgatgatgaaattgagacatggatcagagatgcaatccttcacggttattggagtgttcttgagcatagcgtttatactttttctatagaagaaatttctcgtgtagcttcacatcagcttgtgaggcataggattgcaagttatacgcaaatgagtcataggtttgctaagcctatagacgaatactacaaaccaattattccgccatctgccaagaaaagaagtaaagagttagtggagaaagcgtataaggaagcctatgacaactattacacacttttagaaagcggtgtaccagaggaagatgcaagatatgtactaccaaatggagtaaatacaaatattgtcgtcacaatgaatgctagagagttatataacttcttctcgcttcgactgtgttcaagggcacagtgggagattagagcaatagcttggaaaatgttagaggaggttaagaaagttcatccccgactattcaaatatactggaccaaattgtataatacatgaaaacttcataagaaatgaaaatgaatctataactttagaagatatatttaaagattacaaattagagttcatatctcaaagatgcatcgagggagtattgagagacggaataaaaaaatgcatcataaattcgcgttctgtattggataatattaaa

SEQ ID NO: 54 - DNA, 951 bp, Aquifex aeolicus
ttgatgaaaatctacttaatgggctcggaccagaggatagttaggtgtgcgagggtgtcctttgcaaaggactcatatgtcgatgaaaagagggacaaaagactcattcgctacctcttcaagcacaggcacgcgtctccctttgagcacaacatcattgcctttgagtggaaaaaggagaagtggatagaactcctttcaaagctggaaaaccctaccgttcaggtttactactcaaatggctttgtttttcttaacttaaggaacgctatcaacgtatgggaacttttgcccgacgcagtcaaagaaaggataaaggaggcttttcctaccacttacggagtaattcagcggagaggggaaattgaggatgaggaactttattcccttccctacactaaagataaagcttacgtaaaggaaaagattgagacgagttctggctggatcgggcttgttgataagctggaactggaaactgatatggatttttacacttttgtggttgaatgccctctttttgtagcccgtcagtggatgaggcacaggttcggctcttacaacgaggtttctaaaaggtacgtgggaaaggagtttctggaattttatctccctaaatacataagaaagcaagcggagaaaaacaaacaggcttctgtggatgaacctatttctgagagtgaagtttttataaaaaaaatagaaaatttgataagcaaaagcgtaaagctctacgaggagataatagaaaagggcggtgcaaaagagctcgcaaggggagtgcttccccagtttatgaaaacgaggttttactggaccgttccgaggatttctcttgacaacttcataaccctcagaactcacgaaggtgcacaaaaagagataagggaatttgcagaagctataaaggaaatggtaggatacaggggaactgataagaagaacgtcatt

SEQ ID NO: 55 - DNA, 981 bp, Aeropyrum pernix
gtgtccttggcagcgtccctggaaaaagctggcctcggcatctcggtcaggctactcgagtatactggtgatggagagcgtattgtcgctgttgctagtaaggtaagcctctcccgcagcccggcggagaggctgctggccataggggaagatgaagttgagacttggatactcgagacgtttagaaggcagcatttcagcccctgggagcacagcgtctacacattcatggttgagggtctctcgagggtggctagccaccagctagttagacatagggttgctagctacacacaactaagccataggtatagcgagggctacctcagggaggccgccctcaaggcctgcgaatccatcggcctagactgtccatcgaaaccggctgagacggagggcgggcggaaagccgcatacaggctgtactctcaggctctggagagagctgctcgagatttcggggctagcgagaggtttgctatagcggcgaaggccttcgtaataccgcctaccattctggcgaggggtgatggcggtgatggtgtcgtggaggcgtatctacggtctgcagcgatatactatagcctactctcccggggggctaggcgtgaggatgcgcggtatatcctgcccgatgcgctgagaaccagaatcgttgttactatgaatgcacgggagcttatacaggtcttcttccccctgaggatgtgcactagagcccagtgggagatacgccacatagcctggctgctatggcgagagctatccagggttcatccaaggctgttcaggtgggccgggcccagctgtgtgttaagggagaacacccttaggacgacgcccgccagcttatacagctacctggagggcgtagagcggtttacacagccccgctgccctgagcttgttgagaacaaggctataccgggatgcctcaggcaggcggcatcggtggcgccccccggagacggggagtacgaa

SEQ ID NO: 56 - DNA, 798 bp, Halobacterium sp.
atggtaccggcgcgtgggttcggagtgtttttgccaccggcaggcacaccgtctagcatgcgcgtccgtctcctcgaagcgactgagaacccggaggaactcatctgtcagagcgcccgcaacgactacatgtccgactgggtcggtgacacgccactcgacaccgcgatggcgtccgtggacggcgacaccacagacgaaaagctgtcgaacctcatcgcacagctgctcactcgcggccactacggccccttcgagcatccgtcggcgacgttcgccatcgagggtgtgagccggtcgtgtatggcgcagctcactcgccaccgccacgccagcttcgacgtgcagtcgatgcgctacgtcgcgttcgacgacgtcgatccggcggcggttgcggagggcgagctggtggtgacgccgccgtcggcgaccgaccccgactgggtcggccgcaaccaggacgcgggcgacatcgacgaggagaccatggccgaacgcgaggcggtcttccaggcgtcggtgcggcgcgccgtcgaagactaccaggaactgctggggctcgggatgcccccggaggacgcccggttcgtgctccccatcgggaccgaagtgaacgtggtgatcacgctgaacccgcggtcgctgatgcacgtcgcggacatgcgggccgccgccgacgcacagtgggagatccgcgagctcaccgaacagctactcgatgcggccgctcagtggtgtccccacaccttcgaatattacgacgcggagatgaaacaccggaagaaccggctcgcgccc

SEQ ID NO: 57 - DNA, 795 bp, Borrelia burgdorferi
ttgaataaagaatataaaattttggataatggttttttaaaacttattgatttcatgggagatgataggagaatagttaaggctgcaaggatttcttatcgagaagagagcgttaaaagaaaagatgcggaacttattgactatttaataagaaatgggcacacaagcccattggagcaggtggtttttacatttcatgttaaagctccaatatttgttgcaaggcaatggatgaggcatagaacggcaaggattaatgaagtttctggatgctacagcttggcaagagaggaattttatgtccctttagaagaagatttaaagtgtcaaacttctagtaatagctctgaaaaagagtttaagtctttggaaaaattgtcggacaaaataaagcatcatcaaaaacattcttatgagctttatcaagatatgatcaatgctaatattccaaaagaactctcaagaatagttttgcccttaagtttatataccgaatggtattggcaaattgatttaaataatctttttcattttattaaattgcgattagccctagacagtccaaaagaaattaaagaaaattcgccaaaagaaatgcgcgaatatgctaaagcattgataagcatagtaagagaaattgtgcccatcgcttttaacagttttgaaaatcattttttaagaggaaagagattttcccacgaagagataattgcaattattaatgctttggatttaaataagcttagtatggatgctgaaaaattgaacttattgaaagataagctaggaattgat

SEQ ID NO: 58 - DNA, 915 bp, Treponema pallidum
gtgacgttgcgtacgcttcaagccggtgtggcggtcagtatcgctctggatcgtgtgtgctttttctgttataacggggcggtggcacactgtgtagtagaagctgccgaagatattttggaccggcgtttttctgtattggataagggtttcgtgcgtttgatagattacctgggaggggatgcacgcattgtgcaggcagcgcgtgtttcttacggtgcggggactaggactgcgcgtgacgatgcggcgcttatcgattttcttttacgcaataagcatacgtctccttttgagcaggtggtccttaccttccatgtacgtgcaccgatttttgtcgcgcgtcagtggatgcggcatcgcactgctcgcatcagtgaggtgtctagtcgttattcgcttcttagtcatgactgttatgttccgcaggaaacttcagttgcagttcagtccacgcgtaacaagcagggccgcgcgtccgaaggtatctctcctgaacagcagcaggaagtgcgggcagcgtttgaagctcagcagaaagcggcgtgtgccgcttacgacgcattgattcaaaagaacatcgcgcgggagctagcgcgtattaacgtgccgctttcgctttacaccgagtggtattggcagattgatttacacaatctttttcattttttgcgtttacgtgcgagcgctcatgcgcaagcagagattcgtgcgtatgcagaggtaatcattgaaattacccgtgcagttgcgccgtgcgctaccgcctcttttgaaaatcatgaaaaagatggggtgcagttttcagggcgggagtttgctgcgcttaaggccttactggctggagagggtctctcccttgaggggaaggaacgtgcgcgctttgaagaaaaattacgctctggcctgcagcag

SEQ ID NO: 59 - DNA, 1545 bp, Thermoplasma volcanium
atggagttttcgaacgccgagagagatgtattcttaataaaaatagagaaaatgatagacagaggtgccctgatgtcccgttacagtagggcatctgatcctgatatacgatccgtttatgaaaaagagttcaaaacaggggcgaagagcggcgaagagttttacaggaggattttccttgaatatggcgatgaatcgattgccgaacttacaactgcacagatgggcatacagaacgtttcaaacgtagcatccaagataattgaggagataaggatcggcctttcttatcttgaaaaatcgacaagatatgtacgttacgacaaaaaggttgatggcagatacctttacatttcaccagagaggataggaatatcgggggatgatgctaaggattacgtgcaactatgcgacaatctcttcgaattctattccaaggcattgcctcaggtagaagattaccttaggcataagtttccccaagacaagttggtattccaaaatgctggtggaaagactctaaccgaaatggatcaaaacgagaaaaaaatagcagaaagatcctatatcaatgcagttaggtcacgtgcgctggatgatgtaaggtatattttgccagcatcgacgctgacgaacatcggtattagcggcaacggaagggccctaatccatcttatacagaagttaatggaatatgaaatcccagaaactacaaagcttgcaaaggacatatacgatgaactaaagccagaattgccacagcttatagatgatgcactatctggccacggccttgagatcataaattttaagaagaatctcatgggcttgtttccctatgatctcactggcaattttgaaagaattcgcctactctcatatggcgaagaggataaagagctcaggaaggtggcatccctcatcgaatatccattccatggggacgcggcttcactttactcccgcagctctgaatcctacgtaaaatatatgaaagagctcatagagtcgataaggtcgttgagggcaaacaggaggatgaaaccaggaagggcattcgaatcagtaaattatgtttttgagctaaacttaaactacggctcattccgtgatctacaaagacaccgctttcttggtataattaggaaaccgcttacggcagcatacggatacgacactcctccagtgatctctgctatagatgaattgaaaacgcagtatgatgagttgatggcgaattcttcttccttttaccaaaggctaagggaaaagtatgggccctggatatcacagtatgttgttccgttcgcttttaaatacccgatcacgttttccacgaatctctctgaagtaacgtattttgtagaactcaggagtactgcgcaggcccatttcgatctaagagacatagcggtaagcatgtacagggaagtctcgaaagtgcacccaactctgtccagaatcataaaatttgttgatactgcagattatccgcttggcaggttatctgctgagtttaggaaggaatcgaagaaggccggtatt

SEQ ID NO: 60 - DNA, 732 bp, Pyrococcus abyssi
atggttcgagttacgctcgtcaattacacaaggagacccctcgaaacgataacatgggcggccctcgtaagttactgggacgagtggagcactgagtcatttgaaaagatcaacgaggatgatgtaaaagctcacctccccaggatacttggttatggacacgagagcattttagagcacgcaacgttcacattctcaatagagggctgcagtagggtttgcacgcaccagttggtgaggcataggatagccagctacactcagcaaagccaacgctacattgttctcaacgaggagaacgttgaagaaaccttcgtgataccagagtcgataaagaaggacagagaactctatgaaaaatggaagaaagctatggcggagacaataaagctctacaaggaaagtttaaagagaggtattcaccaagaagatgctaggttcatccttccccaggcggttaggagtaaaatagtcgttacgatgaaccttagggagctcaagcacttcttcggcttaagattgtgcgagagggcacagtgggaaatcagggaagttgcctggaagatgcttgaggaaatcgcaaagagggaagagctaaggcctataataaagtgggctaagcttgggcctcgttgtattcagttaggttattgccccgaaagggaattaatgcccccaggatgcttcaagagaacaagggagagatggatgaaacttctcgaaaaaccctta

SEQ ID NO: 61 - DNA, 1587 bp, Chlamydia trachomatis
atgttgagcaaagagggtggtttttctgaggagcaaagagcgcgtttatcgcattttgtgacgaatttagactcgcctatatttgctttgaaaaaccttccagaagtggttaaaggcgctttattttcaaaatattccagatcgactctggggttgcgagcgcttcttttgaaagaatttttagatggggaaggcggtaatttccttgatgatgaccaacaagattgtgagttggggatccaaaaagctgcggacttctatcgtcgcgttttagacaactttggtgatgattctgttggagagttgggaggagcgcatcttgctctggaacaagtatccatgctcgcagcaaaaattttagaagatgctcggattggagggtcccccctagaaaaatcgtctagatacgtttatttcgatcaaaaagttaacggggagtatttatattaccgagaccctattttgatgacctcggcctttaaagacgtctttttggatacttgtgatttcctattcaacacatactccgatcttatccctcaggttcgttcccatttcgagaaactataccctaaagatccagaagtttctcaatcagcgtatacagtttctttacgagctaaagtattagactgtttacgaggtttgctacctgcagcgacactcacaaatttaggtttttttggtaatggccggttttggcagaacttgctacaccgtttgcaagacaatagtttggttgaggtacgcaatattggagagcagtccttaacagaattaatgaaaataattccctcttttgtaagccgcgcagagtctcatcattatcatcaccaagctatggtggattaccgtcgggctttaaaagaacaattaaaaagttttgcacatcgttacggggaagagagagaaatttcgaaagaggctggtgtaaaattagtatacggagatccagacgggttatacaaaattgctgcagcctacatgttcccctactcggaacacacttatgcagagctgttagatatttgtcgcaatattcctaatgaagatctcatgcgtatcttagagtcgggagcttctttccgagagaatcggcggcacaaatcccctcgcggattggaatgtgctgagtttgcttttgatattacagcggattttggagcctatcgggatttacaaagacatcgtatcctaactcaagaaagacagcttttgacgacaaaattgggttacacgatgccttcacaattgatcgacactcctatggaagctcccttcagagaagctatggaaaaagctgatcaagcgtatcgtctaatagcagaagagttcccagaagaagcacaatatgtggttcctttagcttacaatattcgatggcttttccatatcaacgctagaggtttgcagtggctttgtgagttacgctctcaaccacaagggcatgaaagctatcgtaaaattgctatagatatggctagagaggttattcagtttcatccagcttacgagctgttcttgaagtttgtcgactactcagagactgacctaggaagattacaacaagaatcgcgtaaaaagtct

SEQ ID NO: 62 - DNA, 1481 bp, Dictyostelium discoideum
atgggtcttgatattcaaacagaaatcgataaaattgtaattgaaaaggttaaaccagaagttgaatactatgacgttatgggagggtcacatagatgggaagtcaaagtgcacgaccatggtaaagttgcattagtcgatactatgccaagattagcaccagttggtcaaaccgccgatttctcaatttgtcaagcagcaagagtgtcatatggtgccggtactaaaaaagtcactgaagataaaggtttaattcgttatctttatagacatcaacatacttgtaagtatataaataaataaataaataatgaaaaaaaaaaaaaaaaaaaaaaaaaagtttattaacaatttttaatttttaattttttagcaccatttgaaatggtagaatttaaatttcattgtgtaatgccagtatttatcgcacgtcaatggattagacatagaacagcaaatgtcaatgaatatagtgcacgttattcagtattaccagataaattttatcatccatcaattgaggaagttagaaagcaatcaacatcaaatagacaaggtggtgaagaagcattggagccaaaaaccgcccaagagtttttagactatttagataaagttgaagaaaactataaaacctataatgaactcttagagaaaggtttatcacgtgaattgggtcgtattgggttaccagttagtatctacactgaatggtattggaagatcgatttacataatcttttccatttcttaagacttcgtatggatagtcattctcaaaaagagattagagattatgcaaatacaatatttgctctcattcgtccaattgtaccagttgcttgtgaaggcatttatagattatgcttttgaaagtttaaacttacacgtttagaaattgaagcattcgtactggtagtccactcaatactacaaataaaagagaaatagaagaatttgaagaaaagaagaaattattattcccaaatactcaagcctaaaaaaaacacaaaacacaaacacaattatttaattaattaaataatctcatccttttttgtatataaaataaataaaacaaatattattaatgtattgttcattttgttatgaaacaattcattttctaaataaaatatatatgttatcacaataaaattatttatttatatttattttttaaaaaattaatcatcaagaatgatttcagaatcatcttggtattgttgttgttgaggattttgttgttgttgaatgctattattattacgttgatttaattgttctttttgtaattcttctaaatttccagaataggttgaaacatccaattcttttaaagtttcatttgtaatttcagtatcttgggttattatttttattaaattatcatagattttattttgtggattatcaaatgctggtggtaaattggataatggactgtttgaatttggtaatattcaatatgtattctttac

SEQ ID NO: 63 - DNA, 669 bp, Roseophage
atgacacagattgaagcaacatacatcgaccacatgggttcagacctttcagtcgtcaacgcagcacgggttagctttggtaagaagagtgagtgggtttactgtggtcagtcagacggcagagacaaaggtttatctggccgtgacaccaagctcatcaagtacctagccaagcacaagcacatcagccccttcggacatgccttcgcaagcttccacgttaaggctccaatctttgtagcacggcagttggtgaagcataagttcctacgttggaatgagatcagccgccgttacgttgatgatgaacctgagttctacacacctgatgtatggcgtggacgtagtgctgacaagaagcagggtagtgatggtgtagttaacccagagtataacccccaatacctagacaacaaaatcaagtttgcctatctacaagcacttgacataggtatttcacctgaacaagcacggatgctgttgccgcagtccacaatgactgagtggtattggtcaggtagccttgatgcctttgctgacatgtgccgccttcgttgtaaggaagacacacagtatgagagccgtgttgtggctgaccagatcagtgagaagatggcagacctgtatcctgtctcatgggctgcacttatggaaggagaaaagcaatga

SEQ ID NO: 64 - DNA, 650 bp, Chorella virus
atgtccgcaaagctcatttccgttaccaagccagtcgtggagggtgttaacactgcagaggaactaattgcgtatgctgcccgtgtctccaaccccgaaaaccaaattaacaacaagactgcatcagggcttctaaagtattgtatccgccacaagcattggtcaatctttgagactgcgttcatgactcttgaactgaagacgtctcgcggtatcgcagctcaggttcttcgccatcggagcttccacttccaggaattttcccagaggtacgcatctgtgatggaaactccaccacctcatcaagcacgattccaagatcataaaaatcgccaaaattctctggacaccgtcccggaagatgatcaaacgtggtgggcaaccgaacaagaaaaactgtatgcacagagcatggagctctataacaaggctctcgaaaagggaattgcaaaagaatgtgcaaggtttattcttcctctgagtacaccaactactatttacatgtcgggtacgatcagggattggatccattacatcgaactgcgcacttcaaacgggacacaacgagaacacattgatcttgcaaatgcttgcaaagaaattttcattaaggaattccccagcattgcaaaagcacttgattgggtctg